SEQUENCE LISTING (37 sequences)

SEQ ID NO: 1 - protein, 120 aa, Artificial sequence
  Synthetic polypeptide
QLQLVESGGGLVQAGGSLRLSCAASGRTSSMYAMGWFRQSPGNEREFVAGIGWENNTPYYARSVEGRFTISRDNVKNTVFLQMNRLKPEDAAVYFCAAQIGISGTLGDYWGQGTQVTVSS

SEQ ID NO: 2 - protein, 10 aa, Artificial sequence
  Synthetic polypeptide
GRTSSMYAMG

SEQ ID NO: 3 - protein, 22 aa, Artificial sequence
  Synthetic polypeptide
GIGWENNTPYYARSVEGSEQID

SEQ ID NO: 4 - protein, 11 aa, Artificial sequence
  Synthetic polypeptide
QIGISGTLGDY

SEQ ID NO: 5 - DNA, 360 bp, Artificial sequence
  Synthetic polynucleotide
cagttgcagctcgtggagtcgggaggagggctggtgcaggctgggggctctctgagactctcctgtgcagcctctggacgcacctccagtatgtatgccatgggctggttccgccagtctccagggaacgagcgcgagtttgtagcggggattggctgggagaataataccccatactatgcacgctccgtggagggccgattcaccatctccagagacaacgtcaagaacacggtctttctacaaatgaacagactgaaacctgaggacgcggccgtttatttttgtgcagcccaaatcggaatatccggtacattgggggactactggggccaggggacccaggtcaccgtctcctca

SEQ ID NO: 6 - DNA, 30 bp, Artificial sequence
  Synthetic polynucleotide
ggacgcacctccagtatgtatgccatgggc

SEQ ID NO: 7 - DNA, 51 bp, Artificial sequence
  Synthetic polynucleotide
gggattggctgggagaataataccccatactatgcacgctccgtggagggc

SEQ ID NO: 8 - DNA, 33 bp, Artificial sequence
  Synthetic polynucleotide
caaatcggaatatccggtacattgggggactac

SEQ ID NO: 9 - protein, 229 aa, Artificial sequence
  Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - DNA, 1047 bp, Artificial sequence
  Synthetic polynucleotide
cagttgcagctcgtggagtcgggaggagggctggtgcaggctgggggctctctgagactctcctgtgcagcctctggacgcacctccagtatgtatgccatgggctggttccgccagtctccagggaacgagcgcgagtttgtagcggggattggctgggagaataataccccatactatgcacgctccgtggagggccgattcaccatctccagagacaacgtcaagaacacggtctttctacaaatgaacagactgaaacctgaggacgcggccgtttatttttgtgcagcccaaatcggaatatccggtacattgggggactactggggccaggggacccaggtcaccgtctcctcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 11 - protein, 349 aa, Artificial sequence
  Synthetic polypeptide
QLQLVESGGGLVQAGGSLRLSCAASGRTSSMYAMGWFRQSPGNEREFVAGIGWENNTPYYARSVEGRFTISRDNVKNTVFLQMNRLKPEDAAVYFCAAQIGISGTLGDYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 447 aa, Artificial sequence
  Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 218 aa, Artificial sequence
  Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 120 aa, Artificial sequence
  Synthetic polypeptide
EVQLVESGGGLVQPGGSLRLSCAASGRTSSMYAMGWFRQAPGKGREFVAGIGWENNTPYYARSVEGRFTISRDNVKNTVYLQMNSLRAEDTAVYYCAAQIGISGTLGDYWGQGTQVTVSS

SEQ ID NO: 15 - DNA, 360 bp, Artificial sequence
  Synthetic polynucleotide
gaggtgcagcttgttgaaagtggtggaggtcttgttcaaccagggggctccctcagactgtcttgtgcggcgagcgggcggacatcctctatgtatgcgatgggttggttccgacaggcccccggtaaaggacgggagttcgtagctggcatcggttgggaaaacaataccccttattacgcccggtctgttgaaggtcgatttactataagtcgggacaatgtgaaaaatactgtctatctccaaatgaactctctgcgggccgaagatacagcggtgtactattgtgccgcccaaattggaatcagcggaacattgggtgattattggggccaaggtacgcaagttacagtctcctca

SEQ ID NO: 16 - protein, 120 aa, Artificial sequence
  Synthetic polypeptide
EVQLVESGGGLVQPGGSLRLSCAASGRTSSMYAMGWFRQAPGNEREFVAGIGWENNTPYYARSVEGRFTISRDNVKNTVYLQMNSLRAEDTAVYYCAAQIGISGTLGDYWGQGTQVTVSS

SEQ ID NO: 17 - DNA, 360 bp, Artificial sequence
  Synthetic polynucleotide
gaagtccaactggtcgaaagcggcggcggtctcgtccaacctggaggctctcttaggttgtcatgtgccgcctcaggcagaacatccagcatgtacgcaatgggttggttcagacaggctccggggaacgagcgagaattcgtcgcgggaataggatgggagaacaacaccccatactacgcacgcagtgtggaaggccgattcactattagtcgggataatgttaaaaacacggtctaccttcaaatgaactcccttcgcgcagaggatactgcagtttattattgcgcggcccaaataggtataagtggaacactcggggactactggggccagggaacacaggtaaccgtatcatca

SEQ ID NO: 18 - protein, 120 aa, Artificial sequence
  Synthetic polypeptide
EVQLVESGGGLVQPGGSLRLSCAASGRTSSMYAMGWFRQAPGNEREFVAGIGWENNTPYYARSVEGRFTISRDNVKNTVFLQMNRLRAEDTAVYYCAAQIGISGTLGDYWGQGTQVTVSS

SEQ ID NO: 19 - DNA, 360 bp, Artificial sequence
  Synthetic polynucleotide
gaggtccagttggtagaaagtggtggtgggttggtgcaacccggtggctcattgaggctgtcttgtgctgcgagtggcaggacatcctctatgtatgcgatgggatggttccgacaagctccaggaaacgagcgcgagttcgtagccggaattggttgggaaaacaatacgccctattatgcacggtctgtcgaggggaggttcactatctcacgcgacaacgtcaagaacacagtgtttcttcagatgaaccgactccgggcggaggatacggccgtatattattgcgcagcgcaaatcggtatatccggcactcttggtgactattggggccagggtacacaagtgacagtctcttca

SEQ ID NO: 20 - protein, 120 aa, Artificial sequence
  Synthetic polypeptide
EVQLVESGGGLVQPGGSLRLSCAASGRTSSMYAMGWFRQAPGNEREFVAGIGWENNTPYYARSVEGRFTISRDNVKNTVFLQMNRLKPEDTAVYYCAAQIGISGTLGDYWGQGTQVTVSS

SEQ ID NO: 21 - DNA, 360 bp, Artificial sequence
  Synthetic polynucleotide
gaagtgcaactcgtggagagcgggggcggacttgtccaaccgggagggagtttgagactctcatgcgccgcctctggtagaactagcagcatgtacgctatgggatggttcaggcaggctccagggaacgaacgagaattcgttgcaggcataggatgggaaaacaacaccccatattacgctcggtccgtggaaggacgatttactataagccgggacaatgtaaaaaatactgtctttctccagatgaataggctcaagccggaggatacagcagtttattattgcgctgctcaaattgggattagcgggaccctgggtgactattgggggcagggaacgcaagtgactgtcagttct

SEQ ID NO: 22 - protein, 120 aa, Artificial sequence
  Synthetic polypeptide
ELQLVESGGGLVQAGGSLRLSCAASGRTSSMYAMGWFRQAPGNEREFVAGIGWENNTPYYARSVEGRFTISRDNVKNTVFLQMNRLKPEDTAVYYCAAQIGISGTLGDYWGQGTQVTVSS

SEQ ID NO: 23 - DNA, 360 bp, Artificial sequence
  Synthetic polynucleotide
gagttgcaactggtggaaagtggtggcgggttggttcaggcaggcggttcccttcgcctctcctgtgcggcgagtggacgcacatcatccatgtacgcaatggggtggtttcgacaagcccccggaaacgaacgcgaatttgttgctgggattggatgggaaaacaatacgccgtactatgcccggagcgtagaaggacgattcaccatttccagggacaacgtcaaaaacacggtcttcttgcaaatgaaccgcttgaaaccagaggataccgcagtatactattgtgctgcccagatcggcatatcaggcacactgggcgactattggggccaagggacccaggtcactgtatccagc

SEQ ID NO: 24 - protein, 120 aa, Artificial sequence
  Synthetic polypeptide
ELQLVESGGGLVQAGGSLRLSCAASGRTSSMYAMGWFRQAPGNEREFVAGIGWENNTPYYARSVEGRFTISRDNVKNTVFLQMNRLKPEDAAVYFCAAQIGISGTLGDYWGQGTQVTVSS

SEQ ID NO: 25 - DNA, 360 bp, Artificial sequence
  Synthetic polynucleotide
gaactgcaactcgtagaatctgggggtggcttggtccaggccgggggcagtctgcgactttcctgtgccgcatcaggaaggacctccagcatgtatgcgatgggatggttccgacaagctccgggaaatgagcgcgagtttgttgcgggaattggctgggagaataacacgccctattatgctcggtccgtagaggggaggttcactatcagccgagataatgtaaaaaacaccgtattcctccaaatgaatcggttgaaaccagaggacgcagcggtctacttttgcgccgcgcaaatcggcataagcggtacattgggggattactggggtcaaggcacacaggtaaccgtctctagt

SEQ ID NO: 26 - protein, 349 aa, Artificial sequence
  Synthetic polypeptide
EVQLVESGGGLVQPGGSLRLSCAASGRTSSMYAMGWFRQAPGKGREFVAGIGWENNTPYYARSVEGRFTISRDNVKNTVYLQMNSLRAEDTAVYYCAAQIGISGTLGDYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 1047 bp, Artificial sequence
  Synthetic polynucleotide
gaggtgcagcttgttgaaagtggtggaggtcttgttcaaccagggggctccctcagactgtcttgtgcggcgagcgggcggacatcctctatgtatgcgatgggttggttccgacaggcccccggtaaaggacgggagttcgtagctggcatcggttgggaaaacaataccccttattacgcccggtctgttgaaggtcgatttactataagtcgggacaatgtgaaaaatactgtctatctccaaatgaactctctgcgggccgaagatacagcggtgtactattgtgccgcccaaattggaatcagcggaacattgggtgattattggggccaaggtacgcaagttacagtctcctcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 28 - protein, 349 aa, Artificial sequence
  Synthetic polypeptide
EVQLVESGGGLVQPGGSLRLSCAASGRTSSMYAMGWFRQAPGNEREFVAGIGWENNTPYYARSVEGRFTISRDNVKNTVYLQMNSLRAEDTAVYYCAAQIGISGTLGDYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - DNA, 1047 bp, Artificial sequence
  Synthetic polynucleotide
gaagtccaactggtcgaaagcggcggcggtctcgtccaacctggaggctctcttaggttgtcatgtgccgcctcaggcagaacatccagcatgtacgcaatgggttggttcagacaggctccggggaacgagcgagaattcgtcgcgggaataggatgggagaacaacaccccatactacgcacgcagtgtggaaggccgattcactattagtcgggataatgttaaaaacacggtctaccttcaaatgaactcccttcgcgcagaggatactgcagtttattattgcgcggcccaaataggtataagtggaacactcggggactactggggccagggaacacaggtaaccgtatcatcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 30 - protein, 349 aa, Artificial sequence
  Synthetic polypeptide
EVQLVESGGGLVQPGGSLRLSCAASGRTSSMYAMGWFRQAPGNEREFVAGIGWENNTPYYARSVEGRFTISRDNVKNTVFLQMNRLRAEDTAVYYCAAQIGISGTLGDYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 1047 bp, Artificial sequence
  Synthetic polynucleotide
gaggtccagttggtagaaagtggtggtgggttggtgcaacccggtggctcattgaggctgtcttgtgctgcgagtggcaggacatcctctatgtatgcgatgggatggttccgacaagctccaggaaacgagcgcgagttcgtagccggaattggttgggaaaacaatacgccctattatgcacggtctgtcgaggggaggttcactatctcacgcgacaacgtcaagaacacagtgtttcttcagatgaaccgactccgggcggaggatacggccgtatattattgcgcagcgcaaatcggtatatccggcactcttggtgactattggggccagggtacacaagtgacagtctcttcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 32 - protein, 349 aa, Artificial sequence
  Synthetic polypeptide
EVQLVESGGGLVQPGGSLRLSCAASGRTSSMYAMGWFRQAPGNEREFVAGIGWENNTPYYARSVEGRFTISRDNVKNTVFLQMNRLKPEDTAVYYCAAQIGISGTLGDYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - DNA, 1047 bp, Artificial sequence
  Synthetic polynucleotide
gaagtgcaactcgtggagagcgggggcggacttgtccaaccgggagggagtttgagactctcatgcgccgcctctggtagaactagcagcatgtacgctatgggatggttcaggcaggctccagggaacgaacgagaattcgttgcaggcataggatgggaaaacaacaccccatattacgctcggtccgtggaaggacgatttactataagccgggacaatgtaaaaaatactgtctttctccagatgaataggctcaagccggaggatacagcagtttattattgcgctgctcaaattgggattagcgggaccctgggtgactattgggggcagggaacgcaagtgactgtcagttctgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 34 - protein, 349 aa, Artificial sequence
  Synthetic polypeptide
ELQLVESGGGLVQAGGSLRLSCAASGRTSSMYAMGWFRQAPGNEREFVAGIGWENNTPYYARSVEGRFTISRDNVKNTVFLQMNRLKPEDTAVYYCAAQIGISGTLGDYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - DNA, 1047 bp, Artificial sequence
  Synthetic polynucleotide
gagttgcaactggtggaaagtggtggcgggttggttcaggcaggcggttcccttcgcctctcctgtgcggcgagtggacgcacatcatccatgtacgcaatggggtggtttcgacaagcccccggaaacgaacgcgaatttgttgctgggattggatgggaaaacaatacgccgtactatgcccggagcgtagaaggacgattcaccatttccagggacaacgtcaaaaacacggtcttcttgcaaatgaaccgcttgaaaccagaggataccgcagtatactattgtgctgcccagatcggcatatcaggcacactgggcgactattggggccaagggacccaggtcactgtatccagcgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 36 - protein, 349 aa, Artificial sequence
  Synthetic polypeptide
ELQLVESGGGLVQAGGSLRLSCAASGRTSSMYAMGWFRQAPGNEREFVAGIGWENNTPYYARSVEGRFTISRDNVKNTVFLQMNRLKPEDAAVYFCAAQIGISGTLGDYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - DNA, 1047 bp, Artificial sequence
  Synthetic polynucleotide
gaactgcaactcgtagaatctgggggtggcttggtccaggccgggggcagtctgcgactttcctgtgccgcatcaggaaggacctccagcatgtatgcgatgggatggttccgacaagctccgggaaatgagcgcgagtttgttgcgggaattggctgggagaataacacgccctattatgctcggtccgtagaggggaggttcactatcagccgagataatgtaaaaaacaccgtattcctccaaatgaatcggttgaaaccagaggacgcagcggtctacttttgcgccgcgcaaatcggcataagcggtacattgggggattactggggtcaaggcacacaggtaaccgtctctagtgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

(19) United States
(12) Patent Application Publication
(10) Pub. No.: US 2025/0326845 A1
(43) Pub. Date: Oct. 23, 2025

(54) SINGLE-DOMAIN ANTIBODY TARGETING PD-1, DERIVATIVE THEREOF AND USE THEREOF

(71) Applicant: LILING BIOPHARMACEUTICALS CO., LTD., Suzhou, Jiangsu (CN)

(72) Inventor: Yujun LI, Suzhou, Jiangsu (CN)

(21) Appl. No.: 18/261,450

(22) PCT Filed: Jan. 13, 2022
(86) PCT No.: PCT/CN2022/071865

§ 371 Date: Jul. 13, 2023

(30) Foreign Application Priority Data
Jan. 14, 2021 (CN) 202110048196.1
Mar. 30, 2021 (CN) 202110343322.6

(51) Int. Cl. C07K 16/28; A61K 39/00; A61P 35/00

(52) U.S. Cl. CPC C07K 16/2818; A61P 35/00; A61K 2039/505; C07K 2317/24; C07K 2317/52; C07K 2317/569; C07K 2317/76; C07K 2317/92

(57) ABSTRACT

Disclosed in the present invention are a single-domain antibody targeting human programmed cell death factor PD-1, and a humanized variant thereof. The VHH chain comprises CDR1 as shown in SEQ ID NO: 2, CDR2 as shown in SEQ ID NO: 3 and CDR3 as shown in SEQ ID NO: 4. The single-domain antibody targeting PD-1 of the present invention is capable of blocking interaction between PD-1 and PD-L1, thus has wide biological application value and clinical application value, and lays a new material foundation for the development of a therapeutic or diagnostic drug targeting PD-1.

### TECHNICAL FIELD

The present invention relates to the field of biotechnology. In particular, the invention relates to single-domain antibodies targeting PD-1, derivatives thereof and uses thereof.

### BACKGROUND

PD-1 (Programmed cell death protein 1, PD-1) and its ligand PD-L1 are important targets of tumour immunity. PD-1 and PD-L1 are a pair of immunosuppressive molecules which are important components of the immune system to prevent autoimmune overstimulation, and the activation of their pathway has effects of suppressing tumour immune response and inducing apoptosis of tumour-specific T cells, and is closely related to tumour development. The use of monoclonal antibodies to block the PD-1 and PD-L1 pathways for treating tumors exhibits good clinical efficacy and safety. A number of antibody-based drugs have been approved for a variety of malignancies, including melanoma, non-small cell lung cancer and advanced renal cell carcinoma, while many ongoing clinical trials are attempting to develop more new indications.

In addition to activated T cells, NKT cells, B cells and activated monocytes, PD-1 is also highly expressed on the surface of depleted T cells. While its ligand PD-L1 is expressed in organs, such as peripheral microvascular endothelial cells, the heart and lungs, in addition to immune cells, such as B cells, T cells and dendritic cells. And PD-L2 is only expressed on the surface of macrophages and dendritic cells. Notably, the expression of PD-L1 has been widely found in human tumors, including human lung, melanoma, ovarian and colon cancers. In both of in vitro and in vivo assays, blocking the PD-L1/PD-1 pathway significantly increased T-cell proliferation, cytokine secretion and T-cell killing on tumor cells. In animals, blockade of the PD-L1/PD-1 pathway significantly blocked tumor growth. Blocking the PD-1/PD-L1 signalling pathway with a PD-1 antibody significantly enhanced tumor-specific T cell expansion and tumor infiltration. The above results demonstrate the important applications of PD-1/PD-L1 blocking antibodies in tumor therapy.

At present, the FDA has approved three PD-1 antibodies for treating a variety of tumors. All of these approved antibody drugs have achieved good therapeutic results in tumor patients. However, overall clinical response rates are still relatively low, typically around 20%. One strategy to improve the efficacy of PD-1/PD-L1 blocking antibodies is to use bispecific antibodies or multi-targeted fusion proteins that simultaneously block other immune-related or unrelated pathways, including the PD-1/PD-L1 pathway, for achieving synergistic and better therapeutic effects. Bispecific antibodies based on the PD-1/PD-L1 pathway are generally in the form of double-chain antibodies. The molecular weight of bispecific antibodies prepared by this method is generally large, so that it is difficult for the antibody to infiltrate tumor tissue and likely to affect clinical outcomes. Double-chain based bispecific antibodies often suffer from mismatch problems in production, thereby causing significant problems in downstream production and purification. Moreover, current PD-1/PD-L1 monoclonal antibodies suffer from problems, such as high dosing and low overall response rates.

The single domain antibody (sdAb) is a special class of antibodies containing only one antibody heavy chain. Similar to the traditional double-chain antibody, it binds selectively to a specific antigen. Single domain antibodies were first identified in camelids and later in Chondrichthyes, such as nurse sharks. The single domain antibody single heavy chain antibody variable region (VHH) is a single functional domain that binds an intact antigen and is only of 12-15 kDa. The simple structure of VHH offers advantages of high specificity, high affinity, low immunogenicity, good permeability when binding to antigens and the ability to reach more obscure targets that cannot be reached by conventional antibodies when used in the treatment of tumors. In addition, single domain antibodies do not create the mismatch problems associated with the fusion of double chain antibodies, since they have only one chain.

### SUMMARY OF THE INVENTION

The purpose of the present invention is to provide a specific single domain antibody targeting PD-1.

Another purpose of the present invention is to provide a specific humanized single domain antibody targeting PD-1.

Still another purpose of the present invention is to provide the use of said single domain antibody or humanized single domain antibody for treating tumors or for preparing drugs for treating tumors.

In the first aspect, the present invention provides a VHH chain of a single domain antibody targeting PD-1, wherein said VHH chain comprises CDR1 shown in SEQ ID NO: 2, CDR2 shown in SEQ ID NO: 3 and CDR3 shown in SEQ ID NO: 4.

In a preferred embodiment, said PD-1 is a human PD-1.

In a preferred embodiment, any one of the above amino acid sequences further comprises a derivative sequence optionally obtained by addition, deletion, modification and/or substitution of at least one (e.g. 1-3, preferably 1-2, more preferably 1) amino acid and capable of retaining binding to PD-1 with high affinity and blocking the binding of PD-L1 to PD-1.

In a preferred embodiment, said VHH chain further comprises the framework regions FR1, FR2, FR3 and FR4, and said FR1, FR2, FR3 and FR4 are shown at positions 1-25, 36-49, 67-98 and 110-120 of the amino acid sequence shown in SEQ ID NO: 1.

In a preferred embodiment, the amino acid sequence of the VHH chain of said single domain antibody targeting PD-1 is shown in SEQ ID NO: 1.

In the second aspect, the invention provides a heavy chain variable region of an antibody targeting PD-1, said heavy chain variable region comprising CDR1 shown in SEQ ID NO: 2, CDR2 shown in SEQ ID NO: 3 and CDR3 shown in SEQ ID NO: 4.

In a preferred embodiment, the amino acid sequence of the heavy chain variable region of said antibody targeting PD-1 is shown in SEQ ID NO: 1.

In the third aspect, the present invention provides a single domain antibody targeting PD-1 having the VHH chain described in the first aspect.

In the fourth aspect, the present invention provides a humanized VHH chain of a single domain antibody targeting PD-1, wherein the framework regions FR1, FR2, FR3 and FR4 are humanized based on the VHH chain described in the first aspect.

In a preferred embodiment, said humanized VHH chains of the single domain antibody targeting PD-1 have the framework regions FR1, FR2, FR3 and FR4 as shown in amino acid positions 1-25, positions 36-49, positions 67-98 and positions 110-120 of the amino acid sequence of SEQ ID NO: 14, or SEQ ID NO: 16, or SEQ ID NO: 18, or SEQ ID NO: 20, or SEQ ID NO: 22, or SEQ ID NO: 24, respectively.

In a preferred embodiment, the amino acid sequence of said humanized VHH chain of the single domain antibody targeting PD-1 is shown in SEQ ID NO: 14, or SEQ ID NO: 16, or SEQ ID NO: 18, or SEQ ID NO: 20, or SEQ ID NO: 22, or SEQ ID NO: 24, respectively.

In the fifth aspect, the present invention provides an antibody targeting PD-1, wherein said antibody comprises one or more VHH chains of a single domain antibody targeting PD-1 as described in the first aspect or a humanized VHH chain of a single domain antibody targeting PD-1 as described in the fourth aspect.

In a preferred embodiment, said antibody targeting PD-1 comprises a monomer, a bivalent antibody, and/or a multivalent antibody.

In the sixth aspect, the present invention provides a bispecific antibody, wherein said bispecific antibody comprises a first antibody and a second antibody, said first antibody comprises a VHH chain of a single domain antibody targeting PD-1 as described in the first aspect, or a heavy chain variable region of an antibody targeting PD-1 as described in the second aspect, or a single domain antibody targeting PD-1 as described in the third aspect, a humanized VHH chain of a single-domain antibody targeting PD-1 as described in the fourth aspect, or an antibody targeting PD-1 as described in the fifth aspect.

In a preferred embodiment, said second antibody may bind the same or a different antigen as the first antibody, or a different epitope of the same antigen as the first antibody.

In a preferred embodiment, said second antibody is a single domain antibody, a single chain antibody or a double chain antibody.

In a preferred embodiment, said bispecific antibody comprises 2-4 single domain antibodies targeting PD-1; preferably, comprising 2 single domain antibodies targeting PD-1; and more preferably, said 2 single domain antibodies targeting PD-1 form a single domain antibody dimer targeting PD-1.

In the seventh aspect, the invention provides a fusion protein, wherein said fusion protein comprises a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, or an antibody targeting PD-1 as described in the fifth aspect, an optional linking sequence, and an Fc fragment of an immunoglobulin.

In a preferred embodiment, said immunoglobulin is IgG1, IgG2, IgG3, IgG4; preferably IgG4.

In the eighth aspect, the invention provides a nucleic acid molecule, wherein said nucleic acid molecule encodes a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect.

In the ninth aspect, the present invention provides an expression vector, wherein said expression vector comprises a nucleic acid molecule as described in the eighth aspect.

In a tenth aspect, the invention provides a host cell, wherein said host cell comprises an expression vector as described in the ninth aspect, or having a nucleic acid molecule as described in the eighth aspect integrated into its genome.

In the eleventh aspect, the present invention provides a method for preparing a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect, wherein said method comprises steps of:
1) culturing a host cell as described in the tenth aspect under suitable conditions, thereby obtaining a culture containing said VHH chain of a single-domain antibody targeting PD-1, heavy chain variable region of an antibody targeting PD-1, single-domain antibody targeting PD-1, humanized VHH chain of a single-domain antibody targeting PD-1, antibody targeting PD-1, bispecific antibody or fusion protein; and
2) optionally, isolating or recovering said VHH chain of a single-domain antibody targeting PD-1, heavy chain variable region of an antibody targeting PD-1, single-domain antibody targeting PD-1, humanized VHH chain of a single-domain antibody targeting PD-1, antibody targeting PD-1, bispecific antibody or fusion protein from said culture.

In the twelfth aspect, the present invention provides an immunoconjugate, wherein said immunoconjugate comprises:
1) a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect; and
2) a conjugated moiety selected from a detectable marker, drug, toxin, cytokine, radionuclide, or enzyme.

In a preferred embodiment, said conjugated moiety is a drug or toxin.

In a preferred embodiment, said immunoconjugate is an Antibody-Drug-Couple (ADC).

In a preferred embodiment, said conjugated moiety is a detectable marker.

In a preferred embodiment, said conjugated moiety is selected from a fluorescent or luminescent marker, a radiolabel, an MRI (magnetic resonance imaging) or CT (computed tomography) contrast agent, or an enzyme capable of producing a detectable product, a radionuclide, a biotoxin, a cytokine (e.g. IL-2, etc.), an antibody, an antibody Fc fragment, an antibody scFv fragment, a gold nanoparticle/nanorod, viral particle, liposome, magnetic nanoparticle, prodrug-activating enzyme (e.g., DT-cardiac flavoprotein (DTD) or biphenylyl hydrolase-like protein (BPHL)), chemotherapeutic agent (e.g., cisplatin), or nanoparticle in any form, etc.

In a preferred embodiment, said immunoconjugate comprises: a multivalent (e.g. bivalent) a VHH chain of a single-domain antibody targeting PD-1 as described in claim 1, a heavy chain variable region of an antibody targeting PD-1 as described in claim 2, a single-domain antibody targeting PD-1 as described in claim 3, a humanized VHH chain of a single-domain antibody targeting PD-1 as described in claim 4, an antibody targeting PD-1 as described in claim 5, a bispecific antibody as described in claim 6, or a fusion protein as described in claim 7.

In a preferred embodiment, said “multivalent” means that a plurality of repeat moieties are included in the amino acid sequence of said immunoconjugate.

In the thirteenth aspect, the present invention provides a pharmaceutical composition, wherein said pharmaceutical composition comprises a therapeutically or diagnostically effective amount of a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect or an immunoconjugate as described in the twelfth aspect, and optionally a pharmaceutically acceptable excipient.

In a preferred embodiment, said pharmaceutical composition is used to treat a tumor, and said tumor is selected from the group consisting of: stomach cancer, liver cancer, leukaemia, kidney tumor, lung cancer, small intestine cancer, bone cancer, prostate cancer, colorectal cancer, breast cancer, colorectal cancer, prostate cancer, cervical cancer, lymphoma, adrenal tumor, or bladder tumor.

In the fourteenth aspect, the invention provides the use of a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect or an immunoconjugate as described in the twelfth aspect in the preparation of:
1) a reagent for detecting PD-1;
2) a reagent for blocking the binding of PD-1 to PD-L1; or
3) a drug for treating tumors.

In a preferred embodiment, said tumor is selected from the group consisting of: stomach cancer, liver cancer, leukaemia, kidney tumor, lung cancer, small intestine cancer, bone cancer, prostate cancer, colorectal cancer, breast cancer, colorectal cancer, prostate cancer, cervical cancer, lymphoma, adrenal tumor, or bladder tumor.

In the fifteenth aspect, the present invention provides a kit, wherein said kit comprises:
1) a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect, an immunoconjugate as described in the twelfth aspect, or a pharmaceutical composition as described in the thirteenth aspect;
2) a container; and
3) optionally, an instruction.

In the sixteenth aspect, the invention provides a method for preparing an antibody targeting an immune checkpoint, wherein said method comprises steps of
a) immunising an animal using immune cells expressing said immune checkpoint; and
b) obtaining an antibody targeting the immune checkpoint from the immunized animal obtained in step 1).

In a preferred embodiment, said immune cells are primary immune cells.

In a preferred embodiment, said method further comprises a step of activating the immune cells in advance.

In a preferred embodiment, said immune checkpoint includes but not limited to: PD-1, CTLA-4, TIM3, LAG3, KIR, GITR, VISTA, 4-1BB, CD28, OX40, ICOS, etc.

In a preferred embodiment, said immune cells are lymphocytes or phagocytes; preferably, said immune cells are T lymphocytes, B lymphocytes, K lymphocytes, NK lymphocytes, plasma cells, granulocytes, mast cells, antigen presenting cells or cells of the mononuclear phagocyte system (e.g. macrophages); and more preferably T lymphocytes.

In a preferred embodiment, said antibody is a polyclonal antibody, a monoclonal antibody, a monodomain antibody; and preferably a monodomain antibody.

In a preferred embodiment, said animal comprises, but is not limited to, a mouse, a rat, a camel, an alpaca, a llama, a rabbit; and preferably a mouse, an alpaca.

In the seventeenth aspect, the present invention provides a method for detecting PD-1 protein in a sample, wherein said method comprises steps of:
1) contacting a sample to be tested with a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect, an immunoconjugate as described in the twelfth aspect; and
2) testing the formation of an antigen-antibody complex, the formation of which indicates the presence of PD-1 protein in the sample.

In the eighteenth aspect, the present invention provides a method for treating a disease, wherein said method comprises administering to a subject in need thereof a therapeutically effective amount of a VHH chain of a single-domain antibody targeting PD-1 as described in the first aspect, a heavy chain variable region of an antibody targeting PD-1 as described in the second aspect, a single-domain antibody targeting PD-1 as described in the third aspect, a humanized VHH chain of a single-domain antibody targeting PD-1 as described in the fourth aspect, an antibody targeting PD-1 as described in the fifth aspect, a bispecific antibody as described in the sixth aspect, or a fusion protein as described in the seventh aspect, an immunoconjugate as described in the twelfth aspect, or a pharmaceutical composition as described in the thirteenth aspect.

In a preferred embodiment, said subject comprises a mammal; and preferably a human.

It should be understood that, within the scope of the present invention, each of the above technical features of the present invention and each of the technical features described specifically in the following (e.g. embodiments) can be combined with each other so as to constitute new or preferred technical solutions, which will not be repeated herein for the conciseness of the specification.

### DESCRIPTION OF DRAWINGS

FIG. 1 shows results of affinity assay of VHH-Fc fusion protein, LL-VHH01-Fc binding PD-1 antigen;

FIG. 2 shows results of bioactivity assay of VHH-Fc fusion protein, LL-VHH01-Fc blocking the binding of PD-L1 to PD-1 protein;

FIG. 3 shows that all of 6 humanized antibodies can bind PD-1 antigen well;

FIG. 4 shows that the 6 humanized antibodies can block the PD-1 and PD-L1 signaling pathways well at the cellular level; and

FIG. 5 shows that 3 representative humanized antibodies can control tumor growth well in animals.

### MODES FOR CARRYING OUT THE INVENTION

The inventors have unexpectedly discovered a class of single domain antibodies targeting PD-1 after extensive and in-depth research. The single domain antibody of the present invention exhibits good binding activity to PD-1 molecules, can block the interaction between PD-1 and PD-L1, and has good anti-tumor activities. Based on this single domain antibody targeting PD-1, the present inventors further developed a series of humanized single domain antibodies, and it was found that these humanized single domain antibodies had similar or better affinity and biological effects than the parent antibody, and had good chemical and serum stability, based on which the present invention was completed.

### Definition on Terms

Terms as used herein have same or similar meanings as those routinely understood by a skilled person. For the sake of clarity, some of these terms are defined below.

### Antibody

As used herein, the term “antibody” or “immunoglobulin” is a heterotetrameric protein of about 150,000 daltons having the same structural characteristics, consisting of two identical light chains (L) and two identical heavy chains (H). Each light chain is linked to the heavy chains through a covalent disulfide bond, whereas the numbers of disulfide bonds between the heavy chains among different immunoglobulin isoforms are different. Each heavy and light chain also has regularly spaced intrachain disulfide bonds. Each heavy chain has a variable region (VH) at one end, followed by multiple constant regions. Each light chain has a variable region (VL) at one end and a constant region at the other end; the constant region of the light chain corresponds to the first constant region of the heavy chain, and the variable region of the light chain corresponds to the variable region of the heavy chain. Special amino acid residues form the interface between the variable regions of the light and heavy chains.

### Single-Domain Antibody

As used herein, “single-domain antibody”, “single domain antibody”, “nanobody”, etc. have the same or similar meanings, and refer to a class of antibody molecules that lack the light chain of the antibody and only have variable regions of the heavy chain. Single-domain antibody is the smallest antigen-binding unit, i.e., the smallest antigen-binding fragment with complete functionality. A single domain antibody (VHH) consisting of only one heavy chain variable region is usually constructed by obtaining an antibody that naturally lacks both the light chain and the constant region 1 (CH1) of the heavy chain, and then cloning the heavy chain variable region of the antibody.

In a specific embodiment, the VHH chain of the single domain antibody targeting PD-1 of the present invention comprises CDR1 shown in SEQ ID NO: 2, CDR2 shown in SEQ ID NO: 3, and CDR3 shown in SEQ ID NO: 4. In a preferred embodiment, the amino acid sequence of the VHH chain of said single domain antibody targeting PD-1 is shown in SEQ ID NO: 1. In said VHH chain, there are also framework regions FR1, FR2, FR3 and FR4, amino acid sequences of which are shown in positions 1-25, 36-49, 67-98 and 110-120 of the amino acid sequence shown in SEQ ID NO: 1.

Based on the VHH chain of the single domain antibody targeting PD-1 of the present invention, the present inventors have also humanized the VHH chain, thereby obtaining a humanized VHH chain of the single domain antibody targeting PD-1. In a specific embodiment, the amino acid sequence of the humanized VHH chain of the present invention is shown in SEQ ID NO: 14, or SEQ ID NO: 16, or SEQ ID NO: 18, or SEQ ID NO: 20, or SEQ ID NO: 22, or SEQ ID NO: 24, respectively. In said humanized VHH chain, framework regions FR1, FR2, FR3 and FR4 are also included, amino acid sequences of which are shown in positions 1-25, 36-49, 67-98 and 110-120 positions of the amino acid sequences shown in SEQ ID NO: 14, or SEQ ID NO: 16, or SEQ ID NO: 18, or SEQ ID NO: 20, or SEQ ID NO: 22, or SEQ ID NO: 24, respectively.

Based on the VHH chain of the single domain antibody targeting PD-1 of the present invention or the humanized VHH chain, the present invention also provides an antibody targeting PD-1 comprising one or more of said VHH chains of the single domain antibody targeting PD-1 or the humanized VHH chain of the single domain antibody targeting PD-1. The present invention also provides a bispecific antibody, said bispecific antibody comprises the first antibody and the second antibody, and the first antibody may be a VHH chain of a single domain antibody targeting PD-1 of the present invention, or a humanized VHH chain. A skilled person may select the second antibody of said bispecific antibody as desired. For example, said second antibody may bind the same or a different antigen from the first antibody; and if the second antibody binds the same antigen as the first antibody, it preferably binds to a different epitope. In a specific embodiment, the second antibody may be a single domain antibody, a single chain antibody or a double chain antibody.

A skilled person can also prepare a fusion protein from a VHH chain of the single domain antibody targeting PD-1 of the present invention or a humanized VHH chain, for example, prepare a fusion protein further comprising the Fc fragment of an immunoglobulin. The fusion protein thus obtained not only possesses biological activities of the VHH chain of a single-domain antibody itself, but also possesses other properties conferred by the Fc fragment of the immunoglobulin, such as prolonged plasma half-life, reduced immunogenicity, improved stability, and the like. In a specific embodiment, said fusion protein comprises a VHH chain of a single domain antibody targeting PD-1 of the invention or a humanized VHH chain, an optional linker sequence, and an Fc fragment of an immunoglobulin. In a specific embodiment, said immunoglobulin is IgG1, IgG2, IgG3, IgG4; and preferably IgG4.

The present invention includes not only complete antibodies, but also fragments, derivatives and analogs of said antibodies. As used herein, the terms “fragment”, “derivative” and “analog” refer to a polypeptide which retains essentially the same biological function or activity as the antibody of the present invention. The polypeptide fragment, derivatives or analogs of the present invention may be (i) a polypeptide having one or more conserved or non-conserved amino acid residues (preferably conserved amino acid residues) being replaced, and such replaced amino acid residues may or may not be encoded by the genetic code, or (ii) a polypeptide having substituents in one or more amino acid residues, or (iii) a polypeptide formed by fusing a mature polypeptide with another compound (e.g., a compound that extends the half-life of the polypeptide, e.g., polyethylene glycol), or (iv) a polypeptide formed by fusion of additional amino acid sequences to this polypeptide sequence (e.g., a leading sequence or a secretory sequence or a sequence used to purify this polypeptide or a proteogenic sequence, or a fusion protein formed with a 6His tag). According to the teachings herein, these fragments, derivatives and analogs fall within the scope of what is known to a skilled person.

The antibody of the present invention refers to a polypeptide having PD-1 protein-binding activity and comprising the CDR regions described above. The term also includes variant forms of a polypeptide having the same function as the antibody of the present invention and comprising the CDR regions described above. These variant forms include, but are not limited to, deletions, insertions and/or substitutions of one or more (typically 1-50, preferably 1-30, more preferably 1-20, and the most preferably 1-10) amino acids, and the addition of one or more (typically up to 20, preferably up to 10, and more preferably up to 5) amino acids to the C-terminus and/or N-terminus. For example, substitutions with amino acids of similar or analogous properties typically do not alter the function of proteins in the art. For another example, the addition of one or several amino acids to the C-terminus and/or the N-terminus does not usually alter the function of the protein either. The term also includes active fragments and active derivatives of the antibody of the present invention. Variant forms of the polypeptide include: homologous sequences, conserved variants, allelic variants, natural mutants, induced mutants, proteins encoded by DNA capable of hybridizing to the DNA encoding the antibody of the present invention under conditions of high or low stringency, and polypeptides or proteins obtained by utilizing antisera against the antibody of the present invention.

In addition to a nearly full-length polypeptide, the present invention includes fragments of single domain antibodies of the present invention. Typically, the fragment has at least about 50 contiguous amino acids, preferably at least about 50 contiguous amino acids, more preferably at least about 80 contiguous amino acids, and the most preferably at least about 100 contiguous amino acids of the antibody of the present invention.

In the present invention, “the conservative variant of the antibody according to the present invention” includes polypeptides which, compared with the amino acid sequence of the antibody according to the present invention, are formed by replacing at most 10, preferably at most 8, more preferably at most 5, most preferably at most 3 amino acid with amino acids having similar properties. These conservative variant may be preferably generated through amino acid replacements as shown in, for example the following table.

| | Representative residues | Preferred residues |
|---|---|---|
| Initial residue | for replacement | for replacement |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also provides a polynucleotide encoding the above antibody, or a fragment or fusion protein thereof. The polynucleotides of the present invention may be in the form of DNA or RNA. The form of DNA includes cDNA, genomic DNA or synthetic DNA. The DNA may be single-stranded or double-stranded. The DNA may be a coding or non-coding chain. A polynucleotide encoding a mature polypeptide of the invention includes: a coding sequence encoding only the mature polypeptide; a coding sequence of the mature polypeptide and various additional coding sequences; a coding sequence (and optionally additional coding sequences) of the mature polypeptide and non-coding sequences.

The term “polynucleotide encoding a polypeptide” may include a polynucleotide encoding such polypeptide, and may further include a polynucleotide with additional coding and/or non-coding sequences. The present invention also relates to a polynucleotides which hybridises to the sequence described above and which has at least 50%, preferably at least 70%, more preferably at least 80% identity with that sequence. In particular, the present invention relates to polynucleotides which can hybridise to the polynucleotides described herein under stringent conditions. In the present invention, “stringent condition” means: (1) hybridisation and elution at lower ionic strengths and higher temperatures, e.g., 0.2×SSC, 0.1% SDS, 60° C.; or (2) hybridisation with a denaturant, e.g., 50% (v/v) formamide, 0.1% calf serum/0.1% Ficoll, 42° C., etc.; or (3) hybridisation occurs only when the identity between the two sequences is at least 90%, preferably more than 95%. Moreover, the polypeptide encoded by a hybridisable polynucleotide has the same biological function and activity as the mature polypeptide.

The full-length nucleotide sequences of the antibodies of the present invention, or fragments thereof, can usually be obtained by PCR amplification, recombinant methods or synthetic methods. One possible method is to synthesise a sequence by synthetic methods, especially when the fragment is relatively short in length. Typically, fragments with long sequences can be obtained by first synthesising a plurality of small fragments and then ligating them together. In addition, the coding sequence of the heavy chain can be fused together with an expression tag (e.g. 6His) to form a fusion protein. Once the sequence has been obtained, recombinant methods can be used to obtain the sequence in large quantities. This is typically performed by cloning the sequence into a vector, transferring the vector into a cell, and then isolating the sequence from the proliferated host cell by conventional methods. The biomolecules (nucleic acids, proteins, and the like) to which the present invention relates, include biomolecules in isolated form.

Nowadiay, it is possible to obtain a DNA sequence encoding a protein of the invention (or a fragment or derivative thereof) entirely by chemical synthesis. Such DNA sequence can then be introduced into various existing DNA molecules (or vectors) and cells known in the art. In addition, mutations may be introduced into the protein sequence of the present invention by chemical synthesis.

The present invention also relates to vectors comprising the appropriate DNA sequences as described above, as well as appropriate promoters or control sequences. These vectors can be used to transform appropriate host cells to enable them to express proteins. The host cell may be a prokaryotic cell, such as a bacterial cell; or a lower eukaryotic cell, such as a yeast cell; or a higher eukaryotic cell, such as a mammalian cell. Representative examples include: bacterial cells, such as Escherichia coli, Streptomyces spp; Salmonella typhimurium; fungal cells, such as yeast; insect cells, such as Drosophila S2 or Sf9; and animal cells, such as CHO, COS7, 293 cells.

The transformation of host cells with a recombinant DNA can be performed by conventional techniques well known to a skilled person. When the host is a prokaryote, such as E. coli, competent cells capable of taking up DNAs can be harvested after an exponential growth period and treated with CaCl₂), using steps well known in the art. Another method is to use MgCl₂. The transformation can also be performed by electroporation if desired. When the host is an eukaryotic, following methods of DNA transfection are available: calcium phosphate co-precipitation; conventional mechanical methods, such as microinjection, electroporation, and liposome packaging.

The obtained transformants can be cultured by conventional methods to express the polypeptides encoded by the genes of the present invention. Depending on the host cells used, the medium used in the culture may be selected from a variety of conventional media. The culture is carried out under conditions suitable for the growth of the host cells. After the host cells have grown to an appropriate cell density, the selected promoter is induced by a suitable method (e.g., temperature shift or chemical induction) and the cells are cultured for an additional period of time.

In the above methods, the recombinant polypeptides may be expressed inside the cell, or on the cell membrane, or secreted outside the cell. If desired, the recombinant proteins can be isolated and purified by various separation methods using their physical, chemical and other properties. These methods are well known to a skilled person. Examples of these methods include, but are not limited to: conventional renaturation treatment, treatment with protein precipitants (salting-out methods), centrifugation, osmotic bacterial breakage, ultra-treatment, ultra-centrifugation, molecular sieve chromatography (gel filtration), adsorption chromatography, ion-exchange chromatography, high-performance liquid chromatography (HPLC), and a variety of other liquid chromatography techniques, and combinations of these methods.

The antibodies of the present invention may be used alone or in combination with or conjugated with a detectable marker (for diagnostic purposes), a therapeutic agent, a PK (protein kinase) modifying portion, or any combination thereof. Detectable markers for diagnostic purposes include, but are not limited to: fluorescent or luminescent markers, radiolabelled markers, MRI (Magnetic Resonance Imaging) or CT (Computerised Tomography) contrast agents, or enzymes capable of producing detectable products.

Therapeutic agents that may be bound or conjugated with antibodies of the present invention include, but are not limited to: 1. radionuclides; 2. biotoxins; 3. cytokines, such as IL-2, etc.; 4. gold nanoparticles/nanorods; 5. viral particles; 6. liposomes; 7. magnetic nanoparticles; 8. drug-activating enzymes (e.g., DT-cardiac dystrophinase (DTD) or biphenyl hydrolase-like protein (BPHL)); 9. therapeutic agents (e.g., cisplatin) or any form of nanoparticles, etc.

### Immune Checkpoint

As used herein, immune checkpoint or immune checkpoint molecule has the same meaning as conventionally understood by a skilled person. It refers to a series of molecules expressed on immune cells that can regulate the degree of immune activation, and play an important role in preventing autoimmunity (abnormalities in immune function that result in attacks on normal cells) from occurring. Therefore, one of the important functions of “immune checkpoint molecules” is to keep the activation of the immune system within a normal range in order to prevent over-activation of the immune system.

Abnormal expression and function of immune checkpoint molecules is one of the important reasons for the occurrence of many diseases. For example, if immune checkpoint molecules are over-expressed or the function of immune checkpoint molecules are too strong, the immune function will be suppressed, the body's immunity will be low, and a person is susceptible to tumors and other diseases. On the contrary, if such immune-suppressing function of the immune checkpoint molecules is poor, the body's immune function will also be abnormal. Tumor cells will express some substances to activate the immune checkpoints, and once the latter are activated, the antigen cannot be presented to the T cells, thereby blocking the process of presenting the antigen in the tumor immune loop, thus suppressing the immune function of T cells, allowing the tumor cells to escape from immune surveillance and thus survive.

Commonly used immune checkpoints include CTLA-4 (cytotoxic T lymphocyte-associated antigen-4), PD-1, TIM3, LAG3, KIR, GITR, VISTA, and 4-1BB.

### Immune Cells

As used herein, immune cells and immune effector cells have the same meaning as conventionally understood by a skilled person, which refer to cells that are involved in or associated with an immune response, including lymphocytes and phagocytes. In a specific embodiment, said immune cells refer to lymphocytes that recognise antigens and thereby generate a specific immune response. Said lymphocytes are mainly T lymphocytes, B lymphocytes, K lymphocytes and NK lymphocytes. In addition to lymphocytes, cells involved in the immune response are plasma cells, granulocytes, mast cells, antigen-presenting cells and cells of the mononuclear phagocyte system (e.g. macrophages).

### Immunogonjugate

The present invention also provides an immunoconjugate comprising a VHH chain of a single domain antibody targeting PD-1 of the present invention, a humanised VHH chain and the like, and a conjugated moiety. In a specific embodiment, said conjugated moiety may be a detectable marker, drug, toxin, cytokine, radionuclide, or enzyme, etc., for diagnostic, detection or therapeutic purposes, and the like.

In a preferred embodiment, said immunoconjugate is an Antibody-Drug-Conjugate (ADC).

### Pharmaceutical Composition

The present invention also provides a composition. Preferably, said composition is a pharmaceutical composition comprising an antibody as described above or an active fragment thereof or a fusion protein thereof, and a pharmaceutically acceptable carrier. Typically, these substances may be formulated in a non-toxic, inert, and pharmaceutically acceptable aqueous carrier medium, wherein the pH is typically about 5-8, and preferably about 6-8, although the pH may vary depending on the nature of the substance to be formulated and the condition to be treated. A formulated pharmaceutical composition can be administered by conventional routes, including (but not limited to): intratumoral, intraperitoneal, intravenous, or topical administration.

The pharmaceutical compositions of the present invention can be used to bind PD-1 protein directly, thereby blocking the interaction of PD-1 and PD-L1. Therefore, the pharmaceutical compositions of the present invention can be used to treat tumors. In a preferred embodiment, said tumor is selected from the following group: gastric cancer, liver cancer, leukaemia, kidney tumor, lung cancer, small intestine cancer, bone cancer, prostate cancer, colorectal cancer, breast cancer, colorectal cancer, prostate cancer, cervical cancer, lymphoma, adrenal gland tumor, or bladder tumor. In addition, the pharmaceutical compositions of the present invention may be used in combination with other therapeutic agents.

The pharmaceutical composition of the present invention comprises a safe and effective amount (e.g., 0.001-99 wt %, preferably 0.01-90 wt %, and more preferably 0.1-80 wt %) of the above described single domain antibody of the present invention (or a conjugate thereof) as well as a pharmaceutically acceptable carrier or excipient. Such carriers include (but are not limited to): saline, buffer, glucose, water, glycerol, ethanol, and combinations thereof. The pharmaceutical formulation should be compatible with the mode of administration. The pharmaceutical composition of the present invention may be prepared into the form of an injection, e.g. prepared by conventional methods using saline or an aqueous solution containing glucose and other excipients. A pharmaceutical composition, such as injection and solution is preferably manufactured under sterile conditions.

The active ingredient is administered in a therapeutically effective amount, e.g., about 10 mg/kg body weight—about 50 mg/kg body weight per day. When using the pharmaceutical composition, a safe and effective amount of the immunoconjugate is administered to a mammal, wherein the safe and effective amount is usually at least about 10 μg/kg body weight and in most cases not more than about 50 mg/kg body weight, and preferably the dose is about 10 μg/kg body weight—about 10 mg/kg body weight. Of course, the route of administration, the patient's health status and other factors should also be taken into account for the exact dose, which are within the skill of the skilled practitioner.

### Detection Method

The present invention also relates to a method for detecting PD-1 protein. Steps of the method are substantially as follows: contacting the sample to be tested with a VHH chain of a single domain antibody targeting PD-1 of the present invention, or a humanised VHH chain, an antibody, a fusion protein or an immunoconjugate, etc.; and subsequently detecting the formation of an antigen-antibody complex, the formation of which would indicate the presence of PD-1 protein in the sample.

### Kit

The present invention also provides a kit comprising a VHH chain of a single domain antibody targeting PD-1 of the present invention, or a humanised VHH chain, an antibody, a fusion protein or an immunoconjugate, etc. In a specific embodiment, said kit further comprises a container, instruction, buffer, and the like.

### Method for Preparing an Antibody Targeting an Immune Checkpoint

During the course of research, the present inventors found that the activities of PD-1 single domain antibodies obtained by directly immunising animals using PD-1 protein were poor. However, when the present inventors used T cells for immunisation, PD-1 single domain antibodies with good activities were obtained.

Based on the above findings, the present invention also provides a particular immunisation method, which will enable the preparation of antibodies targeting an immune checkpoint. Said method comprises immunising an animal using immune cells expressing an immune checkpoint. In a preferred embodiment, an animal can be immunised using immune cells expressing the immune checkpoint after said immune cells are activated. Methods or technical means for activating immune cells are well known to a skilled person.

In the method of the present invention for preparing an antibody targeting an immune checkpoint, the used immune cells are preferably primary immune cells. In the method of the present invention, the immunised animals include, but are not limited to, mice, llamas, alpacas, rats, rabbits; preferably mice, alpacas. The antibody obtained by the method of the present invention may be a polyclonal antibody, a monoclonal antibody, a single domain antibody.

### Advantages of the Invention

1. the single domain antibody targeting PD-1 of the present invention exhibits good binding affinity to PD-1, and thus is able to effectively block PD-1 and PD-L1 binding;
2. the humanised single domain antibody of the present invention is able to retain good binding affinity to PD-1 and exhibits similar, or even better, biological effects than the parent antibody at the in vitro cellular level;
3. the humanised single domain antibody of the present invention has excellent chemical and serum stability; and
4. the single domain antibody targeting PD-1 and the humanised single domain antibody of the present invention have wide biological application value and clinical application value, thereby laying a new material foundation for the development of therapeutic or diagnostic drugs targeting PD-1.

The present invention is further described below in connection with specific embodiments. It should be understood that these embodiments are used only to illustrate the invention and are not intended to limit the scope of the invention. Experimental methods for which specific conditions are not indicated in the following embodiments are generally in accordance with conventional conditions, such as those described in Sambrook et al, Molecular Cloning: a Laboratory Manual (New York: Cold Spring Harbor Laboratory Press, 1989), or in accordance with conditions recommended by the manufacturer. Unless otherwise indicated, percentages and parts are weight percentages and weight parts.

### Example 1: Construction of Phage Display Immune Library

### 1.1 Immunisation of Animals

Frozen human peripheral blood mononuclear cells (purchased from Myotonics) were recovered and the concentration was adjusted to 2*10⁶/ml. And then micro magnetic beads (Thermo Fisher Scientific) loaded with CD3 and CD28 antibodies were added according to the recommended ratio to activate T cells in the PBMCs. After the T cells were activated, the magnetic beads were removed using a magnetic rack; the cells were then collected by centrifugation at 1600 rpm for 5 min; and the cells were frozen in a freezing medium. The freezing medium was RPMI-1640:FBS:DMSO=4:5:1. Temporarily, the frozen cells were stored in liquid nitrogen.

An adult healthy alpaca was selected. The frozen T cells were firstly resuscitated and then the cells were washed once using 1*PBS. The cells were resuspended in a buffer, and then used in the subcutaneous immunisation of the alpaca. The alpaca was then immunised for five times using the same method.

### 1.2 Construction of Phage Library

After the immunisation was completed, 50 mL each of peripheral blood was collected from alpacas after the 4th and 5th immunisation, and mixed to isolate peripheral blood mononuclear cells (PBMCs). Total RNAs were further extracted using RNAiso Plus reagent (Takara, Cat no. 9109). The extracted total RNAs were reverse-transcribed using PrimeScript™ II 1st Strand cDNA Synthesis Kit (Takara, Cat no. 6210A) according to the instructions provided with the kit. DNA fragments of the heavy chain variable region of alpaca were amplified using nested PCR. A total of two rounds of nested PCR were performed.

The vector (pComb3XSS) and the PCR-amplified target fragments were digested separately using SfiI overnight at 50° C., and the target fragments were recovered. The digested PCR products were ligated with the vector at a molar ratio of vector:PCR product=1:3. The vector ligated with a VHH was introduced into competence cells using an electrotransformation method to construct a phage display library of the heavy chain single domain antibody. The cells were plated after the electrotransformation. For testing the insertion rate of the library, 48 clones on the plate were randomly selected for colony PCR. Results showed that the insertion rate reached 100%. The cells were gradient-diluted and plated, and the size of the library was calculated to be 1.0×10⁹. The bacteria library was innoculated into 2× 300 mL 2YT+A+G (Amp: 100 μg/ml, Glu: 1%) medium with an initial OD₆₀₀=0.1-0.2, and incubated at 37° C. and 230 rpm until OD₆₀₀=0.8 or higher. Phage libraries were prepared by adding helper phage M13KO7, (helper phage:bacteria=20:1) according to the OD₆₀₀ value. The phage library was gradient-diluted and plated, and the phage library titer was measured to be 5.76×10¹³ cfu/mL by counting the number of clones.

### Example 2: Screening of Anti-Human PD-1 Single Domain Antibody

The constructed immune library of alpaca was screened for affinity by solid phase screening to obtain a specific phage library.

### 2.1 Affinity Panning

### 2.1.1 Panning

1) the target antigen was diluted with a carbonate buffer at pH 9.6 to a final concentration of 5 μg/mL, and then added to the wells of a microplate reader at 100 μL/well, and coated at 4° C. overnight;
2) the coating solution was discarded and washed for 3 times with PBS. Afterwards, 300 μL of 3% BSA-PBS blocking solution was added to each well and blocked at 37° C. for 1 hour;
3) afterwards, the blocking solution was discarded and washed for 3 times with PBS. 100 μL of phage library was added and incubated at 37° C. for 1 hour;
4) unbound phage was aspirated and the plate was washed for 6 times with PBST and 2 times with PBS;
5) 100 μL of Gly-HCl eluate was added to each well and incubated at 37° C. for 8 min;
6) the eluate was transferred to a 1.5 mL sterile centrifuge tube and quickly neutralised with 15 μL of Tris-HCl neutralisation buffer;
7) 10 μL of the neutralised solution was taken and subjected to a gradient dilution, the phage titer was determined and the panning recovery rate was calculated. The remaining eluate was mixed, amplified and purified for the next round of affinity panning. The panning conditions were changed, and the panning conditions for each round are shown in Table 1.

TABLE 1
Affinity panning condition

| | Antigen Coating | | Amount of | Binding | | Number of |
|---|---|---|---|---|---|---|
| | Concentration | blocking | added library | time | PBST | washing |
| Round | (μg/mL) | solution | (cfu) | (h) | Concentration | with PBST |
| 1 | 5 | BSA-PBS | 2 × 10¹¹ | 1 | 0.1% | 6 |
| 2 | 5 | OVA-PBS | 1 × 10¹¹ | 0.75 | 0.25% | 8 |

### 2.1.2 Library Amplification

After panning, it was necessary to amplify the library with main steps as follows:
1) the panned eluate was mixed with 5 mL of E. coli TG1 culture in the pre-logarithmic growth phase, stood for 30 min at 37° C., and then incubated for 30 min with 220 r/min oscillation;
2) centrifuged at 1000 g for 15 min to remove the supernatant, resuspended in 500 μL of 2×YT and plated on a 200 mm 2×YT-GA plate;
3) E. coli cells were scraped into 10 ml of 2×YT liquid medium, 500 μl of suspension was taken and added into 50 ml of 2×YT liquid medium, shaken at 37° C. for 30 min; M13KO7 helper phage was added according to the ratio of cell:phage=1:20, stood at 37° C. for 30 min, and then incubated for 30 min by shaking at 220 r/min;
4) the culture was dispensed into centrifuge tubes at 25° C., 5000 r/min for 10 min, and the cell precipitate was resuspended in 50 mL of 2×YT-AK Liquid Medium, and incubated overnight at 30° C., 230 r/min with shaking;
5) the overnight culture was centrifuged at 4° C., 10000 r/min for 20 mins, the supernatant was transferred to a new centrifuge tube, 1/5 volume of PEG-NaCl was added, mixed well and placed at 4° C. for more than 2 h;
6) the mixture was centrifuged at 4° C., 10000 r/min for 20 mins, the supernatant was removed, and the precipitate was resuspended in 1 mL of PBS, and 1/5 volume of PEG/NaCl was added, mixed well and placed at 4° C. for more than 1 h;
7) the mixture was centrifuged at 4° C., 12000 r/min, 2 min, the supernatant was removed, and the precipitate was resuspended in 200 μL of PBS, which was the amplification product, determined for the titer, and used for the next round of panning or analysis.

### 2.1.3 Phage Rescue

1) 96 monoclones were randomly picked from the plate of the second round of panned eluent titer with a sterilised toothpick, inoculated into 1 mL of 2×YT-A, and incubated for 8 h at 37° C. with 220 r/min oscillation;
2) 200 μL of the above culture was taken, M13KO7 phage was added at a ratio of cell:phage=1:20, 37° C., and stood for 15 min;
3) the culture was shaken at 220 r/min for 45 min;
4) 800 μL of 2×YT-AK was added, incubated overnight at 30° C. with vigorous shaking;
5) centrifuged on the next day at 12000 rpm for 2 min;
6) the supernatant was taken and used for monoclonal ELISA identification.

### 2.1.4 Identification of Positive Phage Clones

1) PD-1 antigen was diluted in carbonate buffer at pH 9.6 to a final concentration of 2 μg/mL, added to wells of a micro-plate reader at 100 μL/well, and coated at 4° C. overnight;
2) the coating solution was discarded and washed with PBST for 3 times;
3) 300 μL of 5% skimmed milk was added into each well which was blocked at 37° C. for 1 h;
4) each well was washed for 3 times with PBST, and 50 μL of phage culture supernatant and 50 μL of 5% skimmed milk were added into each well and incubated for 1 h at 37° C.;
5) each well was washed for 5 times, and then horseradish peroxidase-labelled anti-M13 antibody (diluted 1:10,000 with 5% skimmed milk) was added at 100 μl/well, and incubated for 1 h at 37° C.;
6) the plate was washed for 6 times with PBST. TMB colour development solution was added for the development at 100 μL/well, 37° C., 7 min;
7) a quenching solution was added to quench the reaction at 50 μL/well, and the optical density was measured at 450 nm.
   2.2 Sequencing of positive clones

Three positive clones were detected. The three positive clones were sent to Suzhou Hongxun Biotechnology Co. for sequencing. One of the clones was sequenced successfully. The sequencing results were analysed to obtain the VHH region of the anti-PD-1 antibody, which was named as LL-VHH01. The CDR region of LL-VHH01 was analysed using Kabat nomenclature, and the corresponding sequences of the CDR regions were obtained. The amino acid sequences of the 0 full-length and the CDR region of LL-VHH01 are shown in Table 2, and the nucleic acid sequences are shown in Table 3.

TABLE 2
Amino acid sequence of VHH and corresponding
amino acid sequence of CDR

| Name | Amino acid sequence | CDR1 | CDR2 | CDR3 |
|---|---|---|---|---|
| LL-VHH01 | QLQLVESGGGLVQAGGSLRLSCAASGR | GRTSSM | GIGWEN | QIGISGTL |
| | TSSMYAMGWFRQSPGNEREFVAGIGWE | YAMG | NTPYYA | GDY (SEQ |
| | NNTPYYARSVEGRFTISRDNVKNTVFL | (SEQ ID | RSVEG | ID NO: 4) |
| | QMNRLKPEDAAVYFCAAQIGISGTLGD | NO: 2) | (SEQ ID | |
| | YWGQGTQVTVSS (SEQ ID NO: 1) | | NO: 3) | |

The amino acid sequence of LL-VHH01 is SEQ ID NO: 1, the sequence of CDR1 is SEQ ID NO: 2, the sequence of CDR2 is SEQ ID NO: 3, and the sequence of CDR3 is SEQ ID NO: 4.

TABLE 3
Nucleic acid sequence of LL-VHH01 and corresponding
nucleic acid sequence of CDR

| Name | Nucleic acid sequence | CDR1 | CDR2 | CDR3 |
|---|---|---|---|---|
| LL-VHH01 | CAGTTGCAGCTCGTGGAGTCGGGAG | GGACGC | GGGATTG | CAAATCG |
| | GAGGGCTGGTGCAGGCTGGGGGCTC | ACCTCC | GCTGGGA | GAATATC |
| | TCTGAGACTCTCCTGTGCAGCCTCT | AGTATG | GAATAAT | CGGTACA |
| | GGACGCACCTCCAGTATGTATGCCA | TATGCC | ACCCCAT | TTGGGGG |
| | TGGGCTGGTTCCGCCAGTCTCCAGG | ATGGGC | ACTATGC | ACTAC |
| | GAACGAGCGCGAGTTTGTAGCGGGG | (SEQ | ACGCTCC | (SEQ ID |
| | ATTGGCTGGGAGAATAATACCCCAT | ID NO: | GTGGAGG | NO: 8) |
| | ACTATGCACGCTCCGTGGAGGGCCG | 6) | GC (SEQ | |
| | ATTCACCATCTCCAGAGACAACGTC | | ID NO: | |
| | AAGAACACGGTCTTTCTACAAATGA | | 7) | |
| | ACAGACTGAAACCTGAGGACGCGGC | | | |
| | CGTTTATTTTTGTGCAGCCCAAATC | | | |
| | GGAATATCCGGTACATTGGGGGACT | | | |
| | ACTGGGGCCAGGGGACCCAGGTCAC | | | |
| | CGTCTCCTCA (SEQ ID NO: 5) | | | |

### Example 3: Preparation of PD-1 Single Domain Antibody VHH-Fc Fusion Protein

The amino acid sequence of the constant region of human immunoglobulin gamma4 (IgG4) was obtained from the database Uniprot (P01861).

(SEQ ID NO: 9)
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

The amino acid sequence of PD-1 single domain antibody VHH and the amino acid sequence of hIgG4-Fc were ligated to obtain the PD-1 VHH-Fc fusion protein, named as: LL-VHH01-Fc. The gene sequence of anti-human PD-1 single domain antibody VHH-Fc fusion protein LL-VHH01-Fc was obtained by gene synthesis.

The gene of PD-1 VHH-Fc fusion protein was then cloned into the expression vector pCDNA4 (Invitrogen, Cat V86220). The PD-1 single domain antibody VHH-Fc fusion protein LL-VHH01-Fc was expressed by using transient transfection of HEK293 suspension cells. Upon the completion of expression, the supernatant was collected and purified using a Protein A affinity chromatography column to ultimately obtain the purified Fc fusion protein LL-VHH01-Fc of the PD-1 single domain antibody.

LL-VHH01-Fc
Gene sequence is:
SEQ ID NO: 10
CAGTTGCAGCTCGTGGAGTCGGGAGGAGGGCTGGTGCAGGCTGGGGGCTC
TCTGAGACTCTCCTGTGCAGCCTCTGGACGCACCTCCAGTATGTATGCCA
TGGGCTGGTTCCGCCAGTCTCCAGGGAACGAGCGCGAGTTTGTAGCGGGG
ATTGGCTGGGAGAATAATACCCCATACTATGCACGCTCCGTGGAGGGCCG
ATTCACCATCTCCAGAGACAACGTCAAGAACACGGTCTTTCTACAAATGA
ACAGACTGAAACCTGAGGACGCGGCCGTTTATTTTTGTGCAGCCCAAATC
GGAATATCCGGTACATTGGGGGACTACTGGGGCCAGGGGACCCAGGTCAC
CGTCTCCTCAGAGTCCAAATATGGTCCCCCATGCCCACCATGCCCAGCAC
CTGAGTTCCTGGGGGGACCATCAGTCTTCCTGTTCCCCCCAAAACCCAAG
GACACTCTCATGATCTCCCGGACCCCTGAGGTCACGTGCGTGGTGGTGGA
CGTGAGCCAGGAAGACCCCGAGGTCCAGTTCAACTGGTACGTGGATGGCG
TGGAGGTGCATAATGCCAAGACAAAGCCGCGGGAGGAGCAGTTCAACAGC
ACGTACCGTGTGGTCAGCGTCCTCACCGTCCTGCACCAGGACTGGCTGAA
CGGCAAGGAGTACAAGTGCAAGGTCTCCAACAAAGGCCTCCCGTCCTCCA
TCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCT
GACCTGCCTGGTCAAAGGCTTCTACCCCAGCGACATCGCCGTGGAGTGGG
AGAGCAATGGGCAGCCGGAGAACAACTACAAGACCACGCCTCCCGTGCTG
GACTCCGACGGCTCCTTCTTCCTCTACAGCAGGCTAACCGTGGACAAGAG
CAGGTGGCAGGAGGGGAATGTCTTCTCATGCTCCGTGATGCATGAGGCTC
TGCACAACCACTACACACAGAAGAGCCTCTCCCTGTCTCTGGGTAAA.
Amino acid sequence is:
SEQ ID NO: 11
QLQLVESGGGLVQAGGSLRLSCAASGRTSSMYAMGWFRQSPGNEREFVAG
IGWENNTPYYARSVEGRFTISRDNVKNTVFLQMNRLKPEDAAVYFCAAQI
GISGTLGDYWGQGTQVTVSSESKYGPPCPPCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK.

### Example 4: Preparation of PD-1 Positive Control Antibody

The amino acid sequence of Pembrolizumab, an anti-PD-1 antibody from Merck Sharp & Dohme (MSD), was obtained from WO2008156712A1, in which the amino acid sequence of the heavy chain is SEQ ID NO: 12.

QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK.

the amino acid sequence of the light chain is SEQ ID NO: 13.

EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

Analogues of Pembrolizumab antibody were also expressed and purified according to the expression and purification method of antibody in the application. The anti-PD-1 antibody analogue from MSD was obtained and renamed as LL-Pos.

### Example 5: Detection of Binding Ability of PD-1 Single Domain Antibody VHH-Fc Fusion Protein to PD-1 Protein by ELISA

Firstly, human PD-1 protein (purchased from Beijing BAPSIS, Cat No. PD1-H5221) was diluted to 1.5 μg/mL with PBS (purchased from Hyclone), and added into 96-well ELISA plate at 100 μl/well for antigen coating, and then incubated in a constant temperature incubator at 37° C. for 60 min. After the incubation, the plate was washed for three times, and PBS with 2% BSA was added at 200 μl/well for blocking, and incubated in a constant temperature incubator at 37° C. for 60 min. At the same time, the sample LL-VHH01-Fc and the control antibody LL-Pos were diluted with a diluent (PBS containing 2% BSA) to 100 μg/ml, and then diluted sequentially in a 3-fold gradient to 0.56 ng/ml in a sample dilution plate. The samples were then added to 96-well ELISA plates and incubated in a constant temperature incubator at 37° C. for 60 min. After the incubation, the plate was washed and the secondary antibody goat anti-human IgG (Fc specific)-HRP antibody (Sigma, Cat No. A-0170) was diluted at 6000-fold with 2% BSA, added to the ELISA plate at 100 μl/well and incubated in a constant temperature incubator at 37° C. for 30 min. After the incubation, the plate was washed for three times and a developing liquid, 100 μg/ml of TMB (3,3′,5,5′-tetramethylbenzidine) was added at 100 μl/well and incubated in a constant temperature incubator at 37° C. for 15 min in darkness. Finally, a quenching solution (2 M/L hydrochloric acid solution) was added at 100 μl/well, the absorbance values were measured at 450 nm/620 nm using a micro-plate reader (Thermo Fisher Scientific, Varioskan LUX), and the data were analysed using Graphpad Prism.

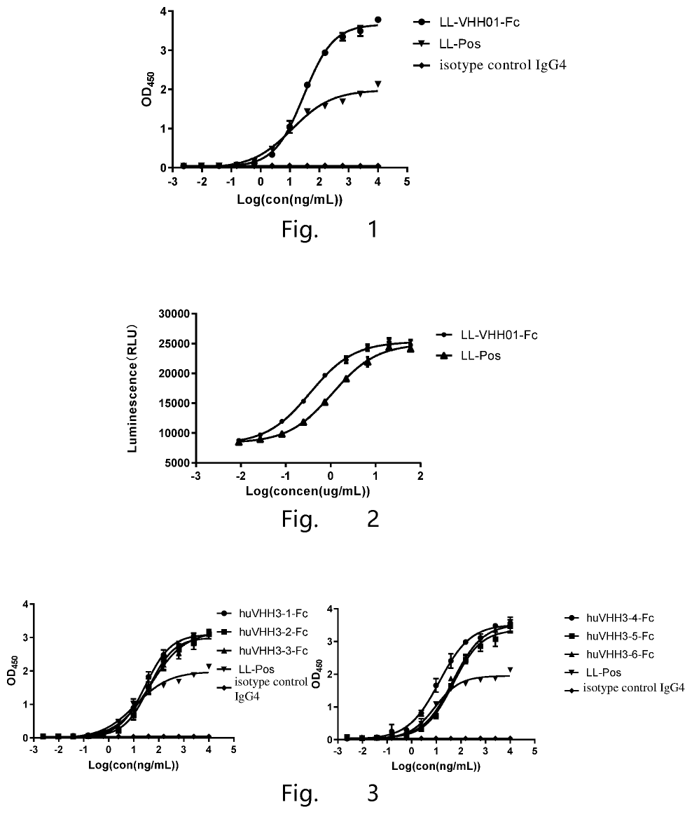

Results are shown in FIG. 1, the EC₅₀ of LL-Pos binding PD-1 protein was 11.84 ng/ml. While the EC₅₀ of LL-VHH01-Fc was 29.69 ng/ml. Under such experimental condition, the upper plateau value of the dose-effect curve of LL-VHH01-Fc binding PD-1 protein was significantly higher than that of LL-Pos. Summing up, the affinity of the VHH Fc fusion protein of the present invention for PD-1 antigen was essentially comparable to that of the control antibody LL-Pos.

### Example 6: Biological Activity of VHH-Fc Fusion Protein Using Luciferase Reporter Gene Assay

CHO/PD-L1 cells (purchased from Promega) were cultured, digested, and resuspended in F-12 Nutrient Mixture Complete Medium containing 10% FBS, and cell density was adjusted to 5×10⁵ cells/ml using Complete Medium based on cell counting results. Afterwards, the cell suspension was transferred to a loading tank and added to a 96-well plate at 100 μl/well using a multichannel pipette and placed in a 5% CO2 incubator at 37° C. for 16-22 h. Meanwhile, Jurkat/PD-1 cells (purchased from Promega) were cultured and cell density was adjusted to 2×10⁶ cells/ml using analytical medium (RPMI 1640 Medium+2% FBS) based on cell counting results. The cell culture plate with CHO/PD-L1 cells was removed from the incubator and 100 μl of culture solution per well was removed using a multichannel pipette, followed by the addition of a sample to be tested, which was gradient-diluted (starting concentration of 1650 nM) at 40 μl/well. Then, the above Jurkat/PD-1 cell suspension was transferred to a loading tank and added into a cell culture plate at 40 μl/well, and then placed in a 5% CO2 incubator at 37° C. for 4-6 h. After the incubation, the cell culture plate was removed and placed at room temperature for 5-10 min, and then 40 μl of One-Glo reagent (purchased from Promega, Cat No. E6130) was added to each well and mixed on a mixer for 5-10 min, and then the chemiluminescence signal value were measured using a multifunctional micro-plate reader (Promega, Cat no. E6130), and the data were analysed using Graphpad Prism.

As shown in FIG. 2, LL-VHH01-Fc can effectively block the binding of PD-1 to PD-L1 and activated the downstream pathway to generate signals, and the EC₅₀ of the dose-effect curve was 0.35 μg/ml. The positive control, LL-Pos, can also effectively block the binding of PD-1 to PD-L1 and activated the downstream pathway to generate signals, and the EC₅₀ of the dose-effect curve was 1.09 μg/ml. ml. The results indicated that the ability of LL-VHH01-Fc to block the binding of PD-1 to PD-L1 was similar to that of the positive control.

The results of FIGS. 1 and 2 indicate that the PD-1 single domain antibody fusion proteins of the present invention are equal to or superior to the already marketed control antibodies in terms of affinity for the antigen PD-1 and blockade of the binding of PD-1 to PD-L1, and thus may be clinically more effective. In addition, single-domain antibodies have no light chain and are well suited as important constituent antibodies for the development of bis or multispecific antibody, which is expected to develop antibody drugs with better efficacy.

### Example 7: Humanisation of Single Domain Antibody

LL-VHH01 was an alpaca-derived antibody, and LL-VHH01 was humanised in order to improve the drugability of this single domain antibody. Steps are listed below:
1. Antibody sequence alignment was performed using IMGT database. According to the results of sequence alignment in the database, IGHV3-23*04 was selected as the parent vector for LL-VHH01 humanisation;
2. CDR regions of LL-VHH01 single domain antibody were transplanted into IGHV3-23*04;
3. The transplanted humanised antibody was reverse-mutated to ensure the affinity of the humanised antibody.

Six candidate humanised antibodies were obtained:

>huVHH3-1
(SEQ ID NO: 14)
EVQLVESGGGLVQPGGSLRLSCAASGRTSSMYAMGWFRQAPGKGREFVAG
IGWENNTPYYARSVEGRFTISRDNVKNTVYLQMNSLRAEDTAVYYCAAQI
GISGTLGDYWGQGTQVTVSS
Nucleic acid sequence:
(SEQ ID NO: 15)
GAGGTGCAGCTTGTTGAAAGTGGTGGAGGTCTTGTTCAACCAGGGGGCTC
CCTCAGACTGTCTTGTGCGGCGAGCGGGCGGACATCCTCTATGTATGCGA
TGGGTTGGTTCCGACAGGCCCCCGGTAAAGGACGGGAGTTCGTAGCTGGC
ATCGGTTGGGAAAACAATACCCCTTATTACGCCCGGTCTGTTGAAGGTCG
ATTTACTATAAGTCGGGACAATGTGAAAAATACTGTCTATCTCCAAATGA
ACTCTCTGCGGGCCGAAGATACAGCGGTGTACTATTGTGCCGCCCAAATT
GGAATCAGCGGAACATTGGGTGATTATTGGGGCCAAGGTACGCAAGTTAC
AGTCTCCTCA
>huVHH3-2
(SEQ ID NO: 16)
EVQLVESGGGLVQPGGSLRLSCAASGRTSSMYAMGWFRQAPGNEREFVAG
IGWENNTPYYARSVEGRFTISRDNVKNTVYLQMNSLRAEDTAVYYCAAQI
GISGTLGDYWGQGTQVTVSS
Nucleic acid sequence:
(SEQ ID NO: 17)
GAAGTCCAACTGGTCGAAAGCGGCGGCGGTCTCGTCCAACCTGGAGGCTC
TCTTAGGTTGTCATGTGCCGCCTCAGGCAGAACATCCAGCATGTACGCAA
TGGGTTGGTTCAGACAGGCTCCGGGGAACGAGCGAGAATTCGTCGCGGGA
ATAGGATGGGAGAACAACACCCCATACTACGCACGCAGTGTGGAAGGCCG
ATTCACTATTAGTCGGGATAATGTTAAAAACACGGTCTACCTTCAAATGA
ACTCCCTTCGCGCAGAGGATACTGCAGTTTATTATTGCGCGGCCCAAATA
GGTATAAGTGGAACACTCGGGGACTACTGGGGCCAGGGAACACAGGTAAC
CGTATCATCA
>huVHH3-3
(SEQ ID NO: 18)
EVQLVESGGGLVQPGGSLRLSCAASGRTSSMYAMGWFRQAPGNEREFVAG
IGWENNTPYYARSVEGRFTISRDNVKNTVFLQMNRLRAEDTAVYYCAAQI
GISGTLGDYWGQGTQVTVSS
Nucleic acid sequence:
(SEQ ID NO: 19)
GAGGTCCAGTTGGTAGAAAGTGGTGGTGGGTTGGTGCAACCCGGTGGCTC
ATTGAGGCTGTCTTGTGCTGCGAGTGGCAGGACATCCTCTATGTATGCGA
TGGGATGGTTCCGACAAGCTCCAGGAAACGAGCGCGAGTTCGTAGCCGGA
ATTGGTTGGGAAAACAATACGCCCTATTATGCACGGTCTGTCGAGGGGAG
GTTCACTATCTCACGCGACAACGTCAAGAACACAGTGTTTCTTCAGATGA
ACCGACTCCGGGCGGAGGATACGGCCGTATATTATTGCGCAGCGCAAATC
GGTATATCCGGCACTCTTGGTGACTATTGGGGCCAGGGTACACAAGTGAC
AGTCTCTTCA
>huVHH3-4
(SEQ ID NO: 20)
EVQLVESGGGLVQPGGSLRLSCAASGRTSSMYAMGWFRQAPGNEREFVAG
IGWENNTPYYARSVEGRFTISRDNVKNTVFLQMNRLKPEDTAVYYCAAQI
GISGTLGDYWGQGTQVTVSS
Nucleic acid sequence:
(SEQ ID NO: 21)
GAAGTGCAACTCGTGGAGAGCGGGGGCGGACTTGTCCAACCGGGAGGGAG
TTTGAGACTCTCATGCGCCGCCTCTGGTAGAACTAGCAGCATGTACGCTA
TGGGATGGTTCAGGCAGGCTCCAGGGAACGAACGAGAATTCGTTGCAGGC
ATAGGATGGGAAAACAACACCCCATATTACGCTCGGTCCGTGGAAGGACG
ATTTACTATAAGCCGGGACAATGTAAAAAATACTGTCTTTCTCCAGATGA
ATAGGCTCAAGCCGGAGGATACAGCAGTTTATTATTGCGCTGCTCAAATT
GGGATTAGCGGGACCCTGGGTGACTATTGGGGGCAGGGAACGCAAGTGAC
TGTCAGTTCT
>huVHH3-5
(SEQ ID NO: 22)
ELQLVESGGGLVQAGGSLRLSCAASGRTSSMYAMGWFRQAPGNEREFVAG
IGWENNTPYYARSVEGRFTISRDNVKNTVFLQMNRLKPEDTAVYYCAAQI
GISGTLGDYWGQGTQVTVSS
Nucleic acid sequence:
(SEQ ID NO: 23)
GAGTTGCAACTGGTGGAAAGTGGTGGCGGGTTGGTTCAGGCAGGCGGTTC
CCTTCGCCTCTCCTGTGCGGCGAGTGGACGCACATCATCCATGTACGCAA
TGGGGTGGTTTCGACAAGCCCCCGGAAACGAACGCGAATTTGTTGCTGGG
ATTGGATGGGAAAACAATACGCCGTACTATGCCCGGAGCGTAGAAGGACG
ATTCACCATTTCCAGGGACAACGTCAAAAACACGGTCTTCTTGCAAATGA
ACCGCTTGAAACCAGAGGATACCGCAGTATACTATTGTGCTGCCCAGATC
GGCATATCAGGCACACTGGGCGACTATTGGGGCCAAGGGACCCAGGTCAC
TGTATCCAGC
>huVHH3-6
(SEQ ID NO: 24)
ELQLVESGGGLVQAGGSLRLSCAASGRTSSMYAMGWFRQAPGNEREFVAG
IGWENNTPYYARSVEGRFTISRDNVKNTVFLQMNRLKPEDAAVYFCAAQI
GISGTLGDYWGQGTQVTVSS
Nucleic acid sequence:
(SEQ ID NO: 25)
GAACTGCAACTCGTAGAATCTGGGGGTGGCTTGGTCCAGGCCGGGGGCAG
TCTGCGACTTTCCTGTGCCGCATCAGGAAGGACCTCCAGCATGTATGCGA
TGGGATGGTTCCGACAAGCTCCGGGAAATGAGCGCGAGTTTGTTGCGGGA
ATTGGCTGGGAGAATAACACGCCCTATTATGCTCGGTCCGTAGAGGGGAG
GTTCACTATCAGCCGAGATAATGTAAAAAACACCGTATTCCTCCAAATGA
ATCGGTTGAAACCAGAGGACGCAGCGGTCTACTTTTGCGCCGCGCAAATC
GGCATAAGCGGTACATTGGGGGATTACTGGGGTCAAGGCACACAGGTAAC
CGTCTCTAGT

### Example 8: Preparation of Six Humanised Antibodies Labelled with Human IgG4 Fc

According to the experimental procedure of Example 3, six humanised antibodies fused with human IgG4 Fc tag were expressed, purified and named as: hu VHH3-1-Fc. hu VHH3-2-Fc, huVHH3-3-Fc. huVHH3-4-Fc, huVHH3-5-Fc, huVHH3-6-Fc. The sequences of six humanised anti-PD-1 single domain antibody fused with hIgG4 Fc are shown as below:

>huVHH3-1-Fc
(SEQ ID NO: 26)
EVQLVESGGGLVQPGGSLRLSCAASGRTSSMYAMGWFRQAPGKGREFVAGIGWENNTPYYARS
VEGRFTISRDNVKNTVYLQMNSLRAEDTAVYYCAAQIGISGTLGDYWGQGTQVTVSSESKYGP
PCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
Nucleic acid sequence:
(SEQ ID NO: 27)
GAGGTGCAGCTTGTTGAAAGTGGTGGAGGTCTTGTTCAACCAGGGGGCTCCCTCAGACTGTCT
TGTGCGGCGAGCGGGCGGACATCCTCTATGTATGCGATGGGTTGGTTCCGACAGGCCCCCGGT
AAAGGACGGGAGTTCGTAGCTGGCATCGGTTGGGAAAACAATACCCCTTATTACGCCCGGTCT
GTTGAAGGTCGATTTACTATAAGTCGGGACAATGTGAAAAATACTGTCTATCTCCAAATGAAC
TCTCTGCGGGCCGAAGATACAGCGGTGTACTATTGTGCCGCCCAAATTGGAATCAGCGGAACA
TTGGGTGATTATTGGGGCCAAGGTACGCAAGTTACAGTCTCCTCAGAGTCCAAATATGGTCCC
CCATGCCCACCATGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCATAATGCC
AAGACAAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAAC
CACTACACACAGAAGAGCCTCTCCCTGTCTCTGGGTAAA
>huVHH3-2-Fc
(SEQ ID NO: 28)
EVQLVESGGGLVQPGGSLRLSCAASGRTSSMYAMGWFRQAPGNEREFVAGIGWENNTPYYARS
VEGRFTISRDNVKNTVYLQMNSLRAEDTAVYYCAAQIGISGTLGDYWGQGTQVTVSSESKYGP
PCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
Nucleic acid sequence:
(SEQ ID NO: 29)
GAAGTCCAACTGGTCGAAAGCGGCGGCGGTCTCGTCCAACCTGGAGGCTCTCTTAGGTTGTCA
TGTGCCGCCTCAGGCAGAACATCCAGCATGTACGCAATGGGTTGGTTCAGACAGGCTCCGGGG
AACGAGCGAGAATTCGTCGCGGGAATAGGATGGGAGAACAACACCCCATACTACGCACGCAGT
GTGGAAGGCCGATTCACTATTAGTCGGGATAATGTTAAAAACACGGTCTACCTTCAAATGAAC
TCCCTTCGCGCAGAGGATACTGCAGTTTATTATTGCGCGGCCCAAATAGGTATAAGTGGAACA
CTCGGGGACTACTGGGGCCAGGGAACACAGGTAACCGTATCATCAGAGTCCAAATATGGTCCC
CCATGCCCACCATGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCATAATGCC
AAGACAAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAAC
CACTACACACAGAAGAGCCTCTCCCTGTCTCTGGGTAAA
>huVHH3-3-Fc
(SEQ ID NO: 30)
EVQLVESGGGLVQPGGSLRLSCAASGRTSSMYAMGWFRQAPGNEREFVAGIGWENNTPYYARS
VEGRFTISRDNVKNTVFLQMNRLRAEDTAVYYCAAQIGISGTLGDYWGQGTQVTVSSESKYGP
PCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
Nucleic acid sequence:
(SEQ ID NO: 31)
GAGGTCCAGTTGGTAGAAAGTGGTGGTGGGTTGGTGCAACCCGGTGGCTCATTGAGGCTGTCT
TGTGCTGCGAGTGGCAGGACATCCTCTATGTATGCGATGGGATGGTTCCGACAAGCTCCAGGA
AACGAGCGCGAGTTCGTAGCCGGAATTGGTTGGGAAAACAATACGCCCTATTATGCACGGTCT
GTCGAGGGGAGGTTCACTATCTCACGCGACAACGTCAAGAACACAGTGTTTCTTCAGATGAAC
CGACTCCGGGCGGAGGATACGGCCGTATATTATTGCGCAGCGCAAATCGGTATATCCGGCACT
CTTGGTGACTATTGGGGCCAGGGTACACAAGTGACAGTCTCTTCAGAGTCCAAATATGGTCCC
CCATGCCCACCATGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCATAATGCC
AAGACAAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAAC
CACTACACACAGAAGAGCCTCTCCCTGTCTCTGGGTAAA
>huVHH3-4-Fc
(SEQ ID NO: 32)
EVQLVESGGGLVQPGGSLRLSCAASGRTSSMYAMGWFRQAPGNEREFVAGIGWENNTPYYARS
VEGRFTISRDNVKNTVFLQMNRLKPEDTAVYYCAAQIGISGTLGDYWGQGTQVTVSSESKYGP
PCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
Nucleic acid sequence:
(SEQ ID NO: 33)
GAAGTGCAACTCGTGGAGAGCGGGGGCGGACTTGTCCAACCGGGAGGGAGTTTGAGACTCTCA
TGCGCCGCCTCTGGTAGAACTAGCAGCATGTACGCTATGGGATGGTTCAGGCAGGCTCCAGGG
AACGAACGAGAATTCGTTGCAGGCATAGGATGGGAAAACAACACCCCATATTACGCTCGGTCC
GTGGAAGGACGATTTACTATAAGCCGGGACAATGTAAAAAATACTGTCTTTCTCCAGATGAAT
AGGCTCAAGCCGGAGGATACAGCAGTTTATTATTGCGCTGCTCAAATTGGGATTAGCGGGACC
CTGGGTGACTATTGGGGGCAGGGAACGCAAGTGACTGTCAGTTCTGAGTCCAAATATGGTCCC
CCATGCCCACCATGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCATAATGCC
AAGACAAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAAC
CACTACACACAGAAGAGCCTCTCCCTGTCTCTGGGTAAA
>huVHH3-5-Fc
(SEQ ID NO: 34)
ELQLVESGGGLVQAGGSLRLSCAASGRTSSMYAMGWFRQAPGNEREFVAGIGWENNTPYYARS
VEGRFTISRDNVKNTVFLQMNRLKPEDTAVYYCAAQIGISGTLGDYWGQGTQVTVSSESKYGP
PCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
Nucleic acid sequence:
(SEQ ID NO: 35)
GAGTTGCAACTGGTGGAAAGTGGTGGCGGGTTGGTTCAGGCAGGCGGTTCCCTTCGCCTCTCC
TGTGCGGCGAGTGGACGCACATCATCCATGTACGCAATGGGGTGGTTTCGACAAGCCCCCGGA
AACGAACGCGAATTTGTTGCTGGGATTGGATGGGAAAACAATACGCCGTACTATGCCCGGAGC
GTAGAAGGACGATTCACCATTTCCAGGGACAACGTCAAAAACACGGTCTTCTTGCAAATGAAC
CGCTTGAAACCAGAGGATACCGCAGTATACTATTGTGCTGCCCAGATCGGCATATCAGGCACA
CTGGGCGACTATTGGGGCCAAGGGACCCAGGTCACTGTATCCAGCGAGTCCAAATATGGTCCC
CCATGCCCACCATGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCATAATGCC
AAGACAAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAAC
CACTACACACAGAAGAGCCTCTCCCTGTCTCTGGGTAAA
>huVHH3-6-Fc
(SEQ ID NO: 36)
ELQLVESGGGLVQAGGSLRLSCAASGRTSSMYAMGWFRQAPGNEREFVAGIGWENNTPYYARS
VEGRFTISRDNVKNTVFLQMNRLKPEDAAVYFCAAQIGISGTLGDYWGQGTQVTVSSESKYGP
PCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
Nucleic acid sequence:
(SEQ ID NO: 37)
GAACTGCAACTCGTAGAATCTGGGGGTGGCTTGGTCCAGGCCGGGGGCAGTCTGCGACTTTCC
TGTGCCGCATCAGGAAGGACCTCCAGCATGTATGCGATGGGATGGTTCCGACAAGCTCCGGGA
AATGAGCGCGAGTTTGTTGCGGGAATTGGCTGGGAGAATAACACGCCCTATTATGCTCGGTCC
GTAGAGGGGAGGTTCACTATCAGCCGAGATAATGTAAAAAACACCGTATTCCTCCAAATGAAT
CGGTTGAAACCAGAGGACGCAGCGGTCTACTTTTGCGCCGCGCAAATCGGCATAAGCGGTACA
TTGGGGGATTACTGGGGTCAAGGCACACAGGTAACCGTCTCTAGTGAGTCCAAATATGGTCCC
CCATGCCCACCATGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTG
AGCCAGGAAGACCCCGAGGTCCAGTTCAACTGGTACGTGGATGGCGTGGAGGTGCATAATGCC
AAGACAAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCCTCCCG
TCCTCCATCGAGAAAACCATCTCCAAAGCCAAAGGGCAGCCCCGAGAGCCACAGGTGTACACC
CTGCCCCCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTACAGCAGGCTAACCGTGGAC
AAGAGCAGGTGGCAGGAGGGGAATGTCTTCTCATGCTCCGTGATGCATGAGGCTCTGCACAAC
CACTACACACAGAAGAGCCTCTCCCTGTCTCTGGGTAAA

### Example 9: Detection of Affinity of Humanised Antibodies for Antigen PD-1

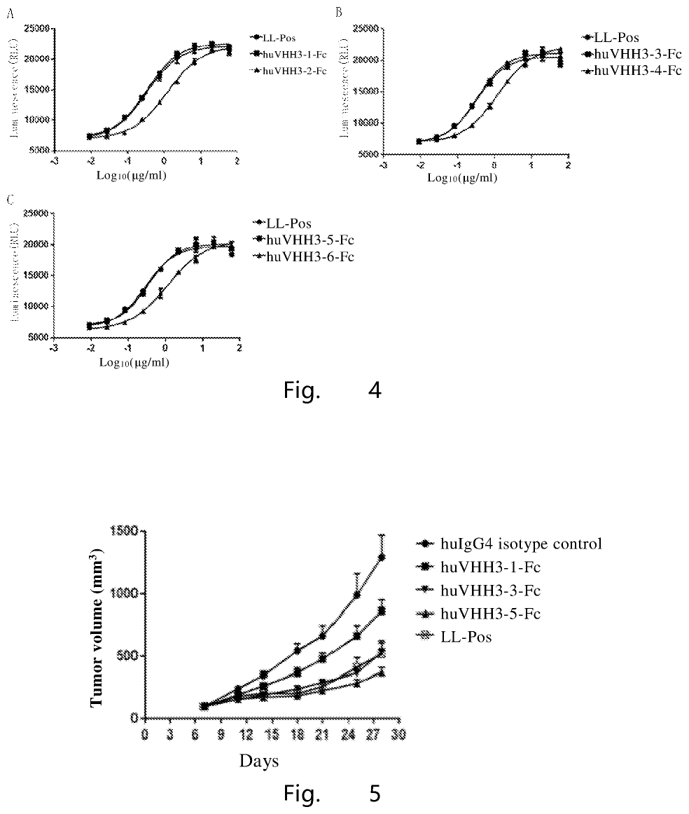

According to the experimental procedure of Example 5, the affinity of the humanised antibody for the antigen PD-1 was tested. The results of the assay are shown in FIG. 3. The results show that the EC₅₀ of huVHH3-1-Fc, huVHH3-2-Fc, huVHH3-3-Fc, hu VHH3-4-Fc, huVHH3-5-Fc, and huVHH3-6-Fc binding to the PD-1 protein were 29.25 ng/ml, 40.50 ng/ml, 46.96 ng/ml, 13.31 ng/ml, 47.61 ng/ml and 45.97 ng/ml, respectively. The experimental results indicated that the affinity of the six humanised antibodies was similar to or better than that of the parent antibody after humanisation.

### Example 10: Detection of Cellular Activity of Humanised Antibodies Blocking PD-1/PD-L1 Signalling Pathway In Vitro

According to the experimental procedure of Example 6, the cellular activity of the humanised antibodies in blocking the PD-1/PD-L1 signalling pathway in vitro was tested. The results of the assay are shown in FIG. 4. The results indicate that the six humanised antibodies exhibited similar or better biological effects than the parent antibodies at the cellular level in vitro after humanisation.

### Example 11: Affinity Determination of Six Humanised Antibodies

An Octet RED 96e molecular interaction analyser from Fortebio was used in this experiment. Candidate antibody molecules were first captured separately (at a concentration of 5 μg/mL) using Protein A probe (Fortebio). The probes were then immersed in a 30 nM or 120 nM human PD-1 antigen solution, respectively, so that the candidate antibody molecules can bind to the antigen for 180 s. Then the probes were immersed in a buffer for a dissociation time of 1400 s, and the binding and dissociation signals were measured. Binding dissociation curves were fitted using the software Octet data analysis software to determine the affinity KD values.

The experiment results are shown in Table 4, which indicate that all six PD-1 single-domain recombinant antibodies can bind the antigen with high affinity and the affinity for binding antigen varies little.

TABLE 4
Binding of six humanised PD-1 recombinant antibodies to antigen

| Antigen | kon (1/Ms) | kdis (1/s) | KD (M) |
|---|---|---|---|
| LL-VHH01-Fc | 7.94E+04 | 2.01E−04 | 2.53E−09 |
| hu-VHH3-1-Fc | 8.40E+04 | 2.40E−04 | 2.86E−09 |
| hu-VHH3-2-Fc | 7.91E+04 | 2.31E−04 | 2.92E−09 |
| hu-VHH3-3-Fc | 8.91E+04 | 2.22E−04 | 2.49E−09 |
| hu-VHH3-4-Fc | 7.45E+04 | 3.08E−04 | 4.13E−09 |
| hu-VHH3-5-Fc | 8.46E+04 | 2.25E−04 | 2.66E−09 |
| hu-VHH3-6-Fc | 9.42E+04 | 2.67E−04 | 2.83E−09 |

### Example 12: In Vivo Pharmacodynamic Evaluation of Humanised PD-1 Single Domain Antibodies

50 6-8-week-old female hPD-1 C57 (PD-1 gene humanized) humanized mice (Baiosetu) were taken, and MC38 WT cells were inoculated into the right subcutaneous side of the mice at a concentration of 1×10⁶ cells/0.1 mL, and when the tumour had grown to approximately 100 mm³, the mice were randomly grouped according to the volume of the tumor, with 5 mice in each group and a total of 8 groups, respectively:

Group 1 KLH IgG4 (0.3 mg/kg) negative control group, Group 2 LL-Pos (0.3 mg/kg) positive control group, Group 3 huVHH3-1-Fc (0.3 mg/kg) treatment group, Group 4 huVHH3-3-Fc (0.3 mg/kg) treatment group, and Group 5 huVHH3-5-Fc (0.3 mg/kg) treatment group.

The administration route for all groups was intraperitoneal injection at a dose of 0.3 mg/kg and a concentration of 0.03 mg/ml. The drug was administered twice a week for 5 consecutive doses, and the experiment was stopped 3 days after the last dose. The tumor volume and body weight were measured twice a week, and mice body weight and tumor volume were recorded. Mice were euthanised at the end of the experiment and the relative tumour inhibition rate TGI %=(1−(Ti−T0)/(Vi−V0))×100% was calculated. (Ti: mean tumor volume of the treatment group or positive control group on day i of drug administration; T0: mean tumor volume of the treatment group or positive control group on day 0 of drug administration; Vi: mean tumor volume of the negative control group on day i of drug administration; V0: mean tumor volume of the negative control group on day 0 of drug administration).

As shown in FIG. 5, on day 28 after the mice were inoculated with tumor cells, the average tumor volume in the negative control group was 1375±115 mm³, and the average tumor volume in the positive control group was 494±267 mm³. The relative tumor inhibition rate was 69.10% compared with that in the negative control group. The average tumor volumes in the treatment Group 3, 4 and 5 were 823±85 mm³, 483±197 mm³ and 320±229 mm³. The relative tumor inhibition rates are 43.30%, 70.00% and 82.75% compared with that in the negative control group, respectively, suggesting that the above humanized anti-PD-1 single-domain antibody was able to in vivo inhibit the growth of subcutaneous transplantation tumors of MC38-WT cells in hPD-1 humanized mouse, and was similar to or significantly superior to the positive control antibody.

### Example 13: Detection on Chemical and Serum Stability of Representative Humanised Antibodies

The candidate molecules were moved into a pH 5.5 buffer (20 mM acetic acid-sodium acetate, 220 mM alginate, 0.02% polysorbate 80), the sample concentration was controlled around 10 mg/ml, a certain volume of sample was dispensed (200 μl/tube) and placed in a 40° C. incubator to investigate the stability at 0, 2 and 4 weeks. Samples were sent and tested according to the time points for the purity of SEC-HPLC and the binding ELISA activity. The results showed that the three candidate molecules had good chemical stability, and there was no significant decrease in monomer purity and binding ELISA activity, as shown in Table 5.

TABLE 5
Detection on chemical stability of three representative humanised antibodies

| | 0 week | | | | 2 week | | | | 4 week | | | |
|---|---|---|---|---|---|---|---|---|---|---|---|---|
| | SEC-HPLC | | | ELISA | SEC-HPLC | | | ELISA | SEC-HPLC | | | ELISA |
| | polymer | monomer | fragment | binding | polymer | monomer | fragment | binding | polymer | monomer | fragment | binding |
| Sample | % | % | % | (%) | % | % | % | (%) | % | % | % | (%) |
| huVHH3-1-Fc | 3.2 | 96.2 | 0.6 | 111.2 | 2.2 | 97.3 | 0.4 | 105.7 | 2.6 | 97.2 | 0.1 | 97.1 |
| huVHH3-3-Fc | 5.1 | 94.5 | 0.4 | 121.3 | 4.0 | 95.6 | 0.4 | 102.9 | 2.9 | 96.9 | 0.2 | 92.9 |
| huVHH3-5-Fc | 6.9 | 92.8 | 0.3 | 93.9 | 3.9 | 95.5 | 0.6 | 103.5 | 2.4 | 97.3 | 0.2 | 86.5 |

The candidate molecules were added into human serum, and the sample concentration was controlled to be around 1 mg/ml, and a certain volume of sample (200 μl/tube) was dispensed and placed in a 37° C. incubator to investigate the stability at 0, 2 and 4 weeks. Samples were sent and tested for the binding ELISA activity according to the time points. The results showed that the three candidate molecules had good serum stability and the binding ELISA activity did not show significant decrease, as shown in Table 6.

TABLE 6
Detection on serum stability for three
representative humanised antibodies

| | 0 week | 2 week | 4 week |
|---|---|---|---|
| | ELISA | ELISA | ELISA |
| Sample | binding(%) | binding(%) | binding(%) |
| huVHH3-1-Fc | 107.5 | 98.7 | 97.8 |
| huVHH3-3-Fc | 99.0 | 92.2 | 101.5 |
| huVHH3-5-Fc | 82.4 | 82.3 | 87.3 |

The above results indicate that the PD-1 humanised single domain antibodies in the present invention have excellent chemical and serum stability for subsequent development.

All documents mentioned in the present invention are cited as references in the present application as if each document was cited individually as a reference. It is further to be understood that after reading the above teachings of the present invention, a skilled person may make various alterations or modifications to the present invention, and these equivalent forms will fall within the scope of the claims appended to the present application.

1. A VHH chain of a single domain antibody targeting PD-1, wherein said VHH chain comprises CDR1 shown in SEQ ID NO: 2, CDR2 shown in SEQ ID NO: 3 and CDR3 shown in SEQ ID NO: 4.

2. A heavy chain variable region of an antibody targeting PD-1, said heavy chain variable region comprising CDR1 shown in SEQ ID NO: 2, CDR2 shown in SEQ ID NO: 3 and CDR3 shown in SEQ ID NO: 4.

3. A single domain antibody targeting PD-1 having the VHH chain of claim 1.

4. A humanized VHH chain of a single domain antibody targeting PD-1, wherein the framework regions FR1, FR2, FR3 and FR4 are humanized based on the VHH chain of claim 1.

5. An antibody targeting PD-1, wherein said antibody comprises one or more VHH chains of a single domain antibody targeting PD-1 of claim 1 or a humanized VHH chain of a single domain antibody targeting PD-1 of claim 4.

6. A bispecific antibody, wherein said bispecific antibody comprises a first antibody and a second antibody, said first antibody comprises a VHH chain of a single domain antibody targeting PD-1 of claim 1, or a heavy chain variable region of an antibody targeting PD-1 of claim 2, or a single domain antibody targeting PD-1 of claim 3, a humanized VHH chain of a single-domain antibody targeting PD-1 of claim 4, or an antibody targeting PD-1 of claim 5.

7. A fusion protein, wherein said fusion protein comprises a VHH chain of a single-domain antibody targeting PD-1 of claim 1, a heavy chain variable region of an antibody targeting PD-1 of claim 2, a single-domain antibody targeting PD-1 of claim 3, a humanized VHH chain of a single-domain antibody targeting PD-1 of claim 4, or an antibody targeting PD-1 of claim 5, an optional linking sequence, and an Fc fragment of an immunoglobulin.

8. A nucleic acid molecule, wherein said nucleic acid molecule encodes a VHH chain of a single-domain antibody targeting PD-1 of claim 1, a heavy chain variable region of an antibody targeting PD-1 of claim 2, a single-domain antibody targeting PD-1 of claim 3, a humanized VHH chain of a single-domain antibody targeting PD-1 of claim 4, an antibody targeting PD-1 of claim 5, a bispecific antibody of claim 6, or a fusion protein of claim 7.

9. An expression vector, wherein said expression vector comprises a nucleic acid molecule of claim 8.

10. A host cell, wherein said host cell comprises an expression vector of claim 9, or having a nucleic acid molecule of claim 8 integrated into its genome.

11. A method for preparing a VHH chain of a single-domain antibody targeting PD-1 of claim 1, a heavy chain variable region of an antibody targeting PD-1 of claim 2, a single-domain antibody targeting PD-1 of claim 3, a humanized VHH chain of a single-domain antibody targeting PD-1 of claim 4, an antibody targeting PD-1 of claim 5, a bispecific antibody of claim 6, or a fusion protein of claim 7, wherein said method comprises steps of:
1) culturing a host cell of claim 10 under suitable conditions, thereby obtaining a culture containing said VHH chain of a single-domain antibody targeting PD-1, heavy chain variable region of an antibody targeting PD-1, single-domain antibody targeting PD-1, humanized VHH chain of a single-domain antibody targeting PD-1, antibody targeting PD-1, bispecific antibody or fusion protein; and
2) optionally, isolating or recovering said VHH chain of a single-domain antibody targeting PD-1, heavy chain variable region of an antibody targeting PD-1, single-domain antibody targeting PD-1, humanized VHH chain of a single-domain antibody targeting PD-1, antibody targeting PD-1, bispecific antibody or fusion protein from said culture.

12. An immunoconjugate, wherein said immunoconjugate comprises:
1) a VHH chain of a single-domain antibody targeting PD-1 of claim 1, a heavy chain variable region of an antibody targeting PD-1 of claim 2, a single-domain antibody targeting PD-1 of claim 3, a humanized VHH chain of a single-domain antibody targeting PD-1 of claim 4, an antibody targeting PD-1 of claim 5, a bispecific antibody of claim 6, or a fusion protein of claim 7; and
2) a conjugated moiety selected from a detectable marker, drug, toxin, cytokine, radionuclide, or enzyme.

13. A pharmaceutical composition, wherein said pharmaceutical composition comprises a therapeutically or diagnostically effective amount of a VHH chain of a single-domain antibody targeting PD-1 of claim 1, a heavy chain variable region of an antibody targeting PD-1 of claim 2, a single-domain antibody targeting PD-1 of claim 3, a humanized VHH chain of a single-domain antibody targeting PD-1 of claim 4, an antibody targeting PD-1 of claim 5, a bispecific antibody of claim 6, or a fusion protein of claim 7 or an immunoconjugate of claim 12, and optionally a pharmaceutically acceptable excipient.

14. Use of a VHH chain of a single-domain antibody targeting PD-1 of claim 1, a heavy chain variable region of an antibody targeting PD-1 of claim 2, a single-domain antibody targeting PD-1 of claim 3, a humanized VHH chain of a single-domain antibody targeting PD-1 of claim 4, an antibody targeting PD-1 of claim 5, a bispecific antibody of claim 6, or a fusion protein of claim 7 or an immunoconjugate of claim 12 in the preparation of:
1) a reagent for detecting PD-1;
2) a reagent for blocking the binding of PD-1 to PD-L1; or
3) a drug for treating tumors.

15. a kit, wherein said kit comprises:
1) a VHH chain of a single-domain antibody targeting PD-1 of claim 1, a heavy chain variable region of an antibody targeting PD-1 of claim 2, a single-domain antibody targeting PD-1 of claim 3, a humanized VHH chain of a single-domain antibody targeting PD-1 of claim 4, an antibody targeting PD-1 of claim 5, a bispecific antibody of claim 6, or a fusion protein of claim 7, an immunoconjugate of claim 12, or a pharmaceutical composition of claim 13;
2) a container; and
3) optionally, an instruction.

16. A method for preparing an antibody targeting an immune checkpoint, wherein said method comprises steps of
a) immunising an animal using immune cells expressing said immune checkpoint; and
b) obtaining an antibody targeting the immune checkpoint from the immunized animal obtained in step 1).
